(19) United States
(12) Patent Application Publication
(10) Pub. No.: US 2025/0179081 A1
(43) Pub. Date: Jun. 5, 2025

(54) COMPOUND AND PRODUCTION METHOD THEREOF, AFX-TYPE ZEOLITE AND PRODUCTION METHOD THEREOF, AND HONEYCOMB STACKED CATALYST

(71) Applicants: OSAKA UNIVERSITY, Suita-shi (JP); N.E. CHEMCAT Corporation, Minato-ku (JP)

(72) Inventors: Takato MITSUDOME, Suita-shi (JP); Yukio TAKAGI, Minato-ku (JP); Kiyohiko SAITO, Minato-ku (JP); Hiroyasu SUZUKA, Minato-ku (JP); Yosuke IMANAKA, Minato-ku (JP); Yasuyuki BANNO, Minato-ku (JP)

(73) Assignees: OSAKA UNIVERSITY, Suita-shi (JP); N.E. CHEMCAT Corporation, Minato-ku (JP)

(21) Appl. No.: 19/000,007

(22) Filed: Dec. 23, 2024

Related U.S. Application Data
(63) Continuation of application No. 17/614,240, filed on Nov. 24, 2021.

(30) Foreign Application Priority Data
May 27, 2019 (JP) 2019-098586
May 27, 2019 (JP) 2019-098745

(51) Int. Cl. C07D 487/04; C01B 39/48

(52) U.S. Cl. CPC C07D 487/04; C01B 39/48

(57) ABSTRACT

A method to prepare a compound of formula (1), or a salt thereof is provided:

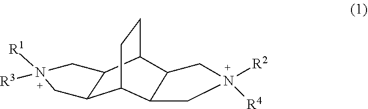

wherein R¹ to R⁴ are each independently an alkyl group.

### CROSS REFERENCE TO RELATED APPLICATIONS

This application is a continuation application of prior U.S. application Ser. No. 17/614,240, filed Nov. 24, 2021, the disclosure of which is incorporated herein by reference in its entirety. U.S. application Ser. No. 17/614,240 is the national stage of PCT/JP2020/018657, filed May 8, 2020, the disclosure of which is incorporated herein by reference in its entirety. U.S. application Ser. No. 17/614,240 claims priority to Japanese Application No. 2019-098745, filed May 27, 2019, and to Japanese Application No. 2019-098586, filed May 27, 2019, the disclosures of which are incorporated herein by reference in their entireties.

### TECHNICAL FIELD

The present invention relates to a compound and a production method thereof, an AFX-type zeolite and a production method thereof, and a honeycomb stacked catalyst.

### BACKGROUND ART

AFX-type zeolites are useful as materials for SCR (Selective Catalytic Reduction) in order to clean up nitrogen oxides in automobile exhaust gases (Non Patent Literature 1). In synthesis of AFX-type zeolites, structure directing agents are used for skeleton structure formation. Structure directing agents are also called OSDAs (Organic Structure Directing Agents), and, for example, N,N,N′,N′-tetraethylbicyclo[2.2.2]oct-7-ene-2,3:5,6-dipyrrolidinium is known. N,N,N′,N′-tetraethylbicyclo[2.2.2]oct-7-ene-2,3:5,6-dipyrrolidinium is a useful compound used as OSDA in preparation of not only AFX-type zeolites, but also MCM-68 zeolites (see, for example, Patent Literatures 1 and 2, and Non Patent Literature 2).

Patent Literature 3 discloses N,N′-diethyl-N,N′-dipropylbicyclo[2.2.2]oct-7-ene-2,3:5,6-dipyrrolidinium and N,N′-diethyl-N,N′-diisopropylbicyclo[2.2.2]oct-7-ene-2,3:5,6-dipyrrolidinium which can be used as OSDA for MCM-70 zeolites.

Patent Literature 4 discloses an AFX-type zeolite having a mesopore, in which the zeolite is controlled with respect to its pore state and thus is excellent in diffusion of a substance and is enhanced in catalyst characteristics. The AFX-type zeolite of Patent Literature 4 is produced without use of any OSDA by a method for crystallizing a composition which includes a silica source, an alumina source, a sodium source and a seed crystal and in which the molar ratio of a quaternary ammonium cation to silica is less than 0.01. Although the AFX-type zeolite of Patent Literature 4 is disclosed to have a mesopore, no AFX-type zeolite having a macropore is known.

### CITATION LIST

### Patent Literature

Patent Literature 1: U.S. Patent Application Publication No. 6049018
Patent Literature 2: Japanese Patent Laid-Open No. 2016-169139
Patent Literature 3: U.S. Patent Application Publication No. 6656268
Patent Literature 4: Japanese Patent Laid-Open No. 2017-128457

### Non Patent Literature

Non Patent Literature 1: S. V. Priya et al., Bull. Chem. Soc. Jpn, 91 (2018) 355.
Non Patent Literature 2: N. Nakazawa et al., Adv. Porous Mater., 4 (2016) 219.

### SUMMARY OF INVENTION

### Technical Problem

Patent Literature 2 discloses use of N,N,N′,N′-tetraethylbicyclo[2.2.2]oct-7-ene-2,3:5,6-dipyrrolidinium as OSDA. However, OSDA, if considered to be utilized as a structure directing agent, is demanded to have the property of providing a zeolite having a desired skeleton structure at a higher efficiency and at a relatively high purity. In addition, OSDA high in process margin in synthesis is desirable from the viewpoint of expansion of industrial applications, and therefore there is a demand for OSDA which can be used in any of various aspects such as a compound and a salt thereof.

One aspect of the present invention has been made in view of the above circumstances, and an object thereof is to provide a novel compound useful as OSDA, and a production method thereof.

Patent Literature 2 discloses a method for producing N,N,N′,N′-tetraethylbicyclo[2.2.2]oct-7-ene-2,3:5,6-dipyrrolidinium as an objective substance by N-ethylation of N,N′-diethylbicyclo[2.2.2]oct-7-ene-2,3:5,6-dipyrrolidine as a precursor. It is here necessary in synthesis of the precursor to use N,N′-diethylbicyclo[2.2.2]oct-7-ene-2,3:5,6-tetracarbodiimide together with a reducing agent which requires any attention in handling and which is very high in reactivity, such as lithium aluminum hydride (LiAlH₄) which is pointed out about ignition properties and explosive properties, and mass production of an objective compound is difficult. OSDA is thus difficult to obtain by a safe procedure, and therefore mass production of an AFX-type zeolite is difficult in nature.

Another aspect of the present invention has been made in view of the above circumstances, and an object thereof is to provide a novel compound which is useful as OSDA and which can be safely and easily synthesized, and a production method thereof.

Still another aspect of the present invention has been made in view of the above circumstances, and an object thereof is to provide a novel AFX-type zeolite and a production method which can efficiently produce the AFX-type zeolite. One different aspect of the present invention has been made in view of the above circumstances, and an object thereof is to provide a honeycomb stacked catalyst using the novel AFX-type zeolite.

It is noted that there is herein no limitation on the objects mentioned and exertion of the effect which is derived from each configuration shown in the Description of Embodiments described below and which cannot be obtained by any conventional art can also be regarded as yet another object of the present invention.

### Solution to Problem

The present inventors have made intensive studies about provision of a compound useful for production of OSDA, and as a result, have found that a certain compound is useful as OSDA, thereby leading to completion of the present invention.

The present inventors have made intensive studies about provision of a compound useful for production of OSDA, and as a result, have found that a certain compound can be safely and easily synthesized and is useful as OSDA, thereby leading to completion of the present invention.

The present inventors have made intensive studies about provision of a compound useful for production of OSDA, and as a result, have found that a certain compound useful as OSDA can be used to thereby efficiently produce an AFX-type zeolite, thereby leading to completion of the present invention.

In other words, the present invention provides various aspects shown below.

[1]

A compound represented by formula (1), or a salt thereof:

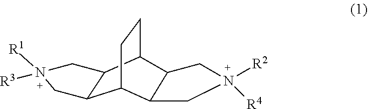

wherein R¹ to R⁴ are each independently an alkyl group.
[2]

A structure directing agent for zeolite synthesis, comprising the compound and/or the salt thereof according to [1].

[3]

A method for producing a compound represented by formula (1), or a salt thereof, the method comprising at least:
a step of providing a compound represented by formula (2); and
a step of N-alkylating the compound represented by the formula (2) with an alkylation reagent;

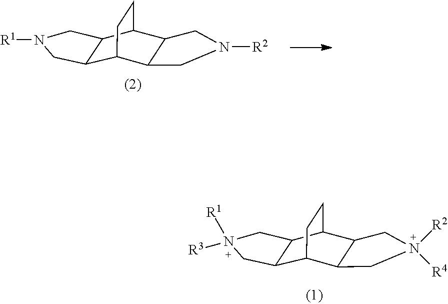

wherein R¹ to R⁴ are each independently an alkyl group.
[4]

An AFX-type zeolite, wherein a compositional ratio except water is represented by the following compositional ratio:
M_{a/b}Q_{c}Si_{48-d}Al_{d}O₉₆
wherein M represents a metal cation, a represents 1 to 10, b represents a valence of M, Q represents a cation derived from the compound and/or the salt thereof according to claim 1, c represents 0.5 to 2, and d represents 4 to 12.
   [5]

The AFX-type zeolite according to [4], wherein X-ray diffraction data comprises the following 2θ values (°): 7.50±0.15, 8.71±0.15, 11.60±0.15, 13.01±0.15, 15.67±0.15, 17.46±0.15, 17.72±0.15, 19.93±0.15, 20.42±0.15, 21.84±0.15, 23.47±0.15, 26.19±0.15, 27.79±0.15, 30.67±0.15, 31.65±0.15, and 33.56±0.15.

[6]

An AFX-type zeolite, wherein
SAR (SiO₂/Al₂O₃ ratio) is 10 or more and 30 or less,
2θ=21.77°±0.15° in an XRD chart obtained by powder X-ray diffraction analysis corresponds to a strongest line, and
an average particle size is 0.6 μm or more.
   [7]

The AFX-type zeolite according to [6], wherein X-ray diffraction data comprises the following 20 values) (°): 7.46±0.15, 8.69±0.15, 11.64±0.15, 12.93±0.15, 15.60±0.15, 17.43±0.15, 17.90±0.15, 19.81±0.15, 20.32±0.15, 21.77±0.15, 23.67±0.15, 26.03±0.15, 28.05±0.15, 30.49±0.15, 31.50±0.15, and 33.71±0.15.

[8]

An AFX-type zeolite, wherein
SAR (SiO₂/Al₂O₃ ratio) is 10 or more and 30 or less,
2θ=21.77°±0.15° in an XRD chart obtained by powder X-ray diffraction analysis corresponds to a strongest line,
an average particle size is 0.6 μm or more, and
a transition metal is supported.
   [9]

A method for producing the AFX-type zeolite according to any one of [4] to [8], the method comprising at least:
a step of preparing a mixture comprising at least:
   a silica and alumina source;
   an organic structure directing agent (OSDA) comprising a compound represented by the following formula (1) and/or a salt thereof:

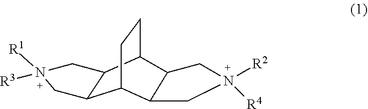

wherein R¹ to R⁴ are each independently an alkyl group;
an alkali metal hydroxide; and
water; and
a step of hydrothermally treating the mixture to synthesize the AFX-type zeolite.
   [10]

A method for producing the AFX-type zeolite according to any one of [6] to [8], the method comprising at least:
a step of preparing a mixture comprising at least:
   a silica and alumina source;
   an organic structure directing agent (OSDA) comprising a compound represented by the following formula (1) and/or a salt thereof:

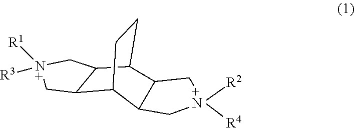

wherein R¹ to R⁴ are each independently an alkyl group;
an alkali metal hydroxide; and
water;
a step of hydrothermally treating the mixture to synthesize the AFX-type zeolite; and
a step of further calcining the AFX-type zeolite obtained, after the hydrothermally treating step.
   [11]

A method for producing the AFX-type zeolite according to [8], the method comprising at least:
a step of preparing a mixture comprising at least:
   a silica and alumina source;
   an organic structure directing agent (OSDA) comprising a compound represented by the following formula (1) and/or a salt thereof:

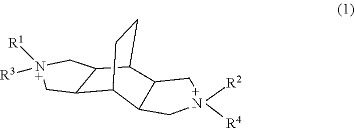

wherein R¹ to R⁴ are each independently an alkyl group;
an alkali metal hydroxide; and
water;
a step of hydrothermally treating the mixture to synthesize the AFX-type zeolite;
a step of further calcining the AFX-type zeolite obtained, after the hydrothermally treating step; and
a step of supporting a transition metal after the calcination step.
   [12]

A method for producing a compound represented by formula (1), or a salt thereof, comprising at least:
a step of providing a compound represented by formula (A) (step I);
a step of reacting the compound represented by the formula (A) with a hydrogen source by use of a catalyst, to thereby obtain a compound represented by formula (2) (step II); and
a step of N-alkylating the compound represented by the formula (2) with an alkylation reagent (step III);

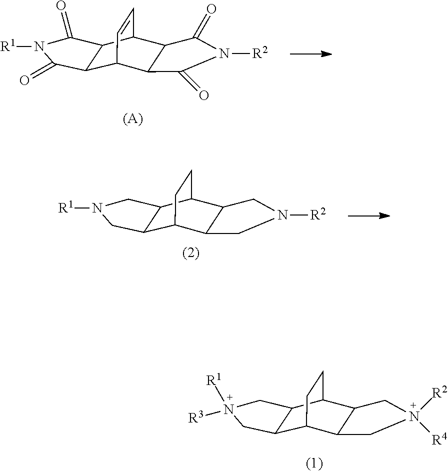

wherein R¹ to R⁴ are each independently an alkyl group.
[13]

A method for producing an AFX-type zeolite, the method comprising at least:
a step of preparing a mixture comprising at least:
   a silica and alumina source;
   an organic structure directing agent (OSDA) comprising a compound represented by the following formula (1) and/or a salt thereof:

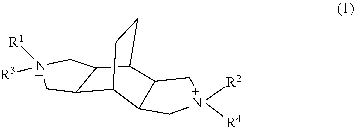

wherein R¹ to R⁴ are each independently an alkyl group;
an alkali metal hydroxide; and
water; and
a step of hydrothermally treating the mixture to synthesize an AFX-type zeolite.
   [14]

The method for producing an AFX-type zeolite according to [13], the method comprising:
a step of reacting N,N′-dialkylbicyclo[2.2.2]oct-7-ene-2,3:5,6-tetracarboxydiimide with a hydrogen source by use of a Pt—V/Z catalyst, to obtain N,N′-dialkylbicyclo[2.2.2]octane-2,3:5,6-dipyrrolidine; and
a step of N-alkylating the N,N′-dialkylbicyclo[2.2.2]octane-2,3:5,6-dipyrrolidine with an alkylation reagent, to thereby obtain the organic structure directing agent (OSDA) comprising a compound represented by formula (1) and/or a salt thereof.
   [15]

An AFX-type zeolite having a macropore.

[16]

A honeycomb stacked catalyst, wherein a honeycomb carrier is coated with the AFX-type zeolite according to [8] or [15].

One aspect of the present invention provides various specific aspects shown below. Hereinafter, any aspect with respect to [A1] to [A18] is also referred to as any “specific aspect in a first group”.

### [A1]

A compound represented by formula (1), or a salt thereof:

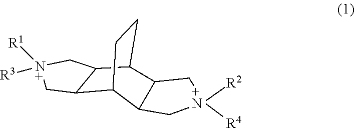

wherein R¹ to R⁴ are each independently an alkyl group.

### [A2]

The compound or the salt thereof according to [A1], wherein R¹ to R⁴ in the formula (1) are the same alkyl groups.

### [A3]

The compound or the salt thereof according to [A1] or [A2], wherein R¹ to R⁴ in the formula (1) are each an ethyl group.

### [A4]

A structure directing agent for zeolite synthesis, comprising the compound and/or the salt thereof according to any of [A1] to [A3].

### [A5]

A method for producing a compound represented by formula (1), or a salt thereof, the method comprising at least:
a step of providing a compound represented by formula (2); and
a step of N-alkylating the compound represented by the formula (2) with an alkylation reagent;

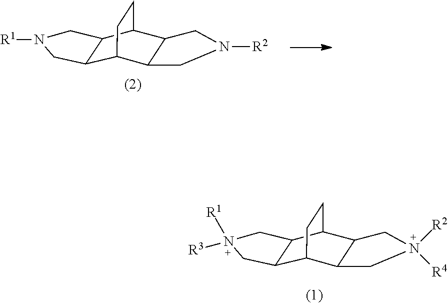

wherein R¹ to R⁴ are each independently an alkyl group.

### [A6]

The production method according to [A5], wherein the alkylation reagent is represented by R′—X, wherein R′ is an alkyl group and X is at least one leaving group selected from the group consisting a halogen atom and a sulfonyl group optionally having a substituent.

### [A7]

The production method according to [A6], wherein the alkylation reagent is an alkyl halide.

### [A8]

The production method according to [A5], wherein
the alkylation reagent is an ethyl halide,
R¹ and R² in the formulae (1) and (2) are each an ethyl group, and
R³ and R⁴ in the formula (1) are each an ethyl group.

### [A9]

An AFX-type zeolite, wherein a compositional ratio except water is represented by the following compositional ratio:
M_{a/b}Q_{c}Si_{48-d}Al_{d}O₉₆
wherein M represents a metal cation, a represents 1 to 10, b represents a valence of M, Q represents a cation derived from the compound and/or the salt thereof according to any of [1] to [3], c represents 0.5 to 2, and d represents 4 to 12.

### [A10]

The AFX-type zeolite according to [A9], wherein X-ray diffraction data comprises the following 20 values) (°): 7.50±0.15, 8.71±0.15, 11.60±0.15, 13.01±0.15, 15.67±0.15, 17.46±0.15, 17.72±0.15, 19.93±0.15, 20.42±0.15, 21.84±0.15, 23.47±0.15, 26.19±0.15, 27.79±0.15, 30.67±0.15, 31.65±0.15, and 33.56 ±0.15.

### [A11]

An AFX-type zeolite, wherein
SAR (SiO₂/Al₂O₃ ratio) is 10 or more and 30 or less,
2θ=21.77°±0.15° in an XRD chart obtained by powder X-ray diffraction analysis corresponds to a strongest line, and
an average particle size is 0.6 μm or more.

### [A12]

The AFX-type zeolite according to [A11], wherein X-ray diffraction data comprises the following 20 values) (°): 7.46±0.15, 8.69±0.15, 11.64±0.15, 12.93±0.15, 15.60±0.15, 17.43±0.15, 17.90±0.15, 19.81±0.15, 20.32±0.15, 21.77±0.15, 23.67±0.15, 26.03±0.15, 28.05±0.15, 30.49±0.15, 31.50±0.15, and 33.71 ±0.15.

### [A13]

The AFX-type zeolite according to [A11] or [A12], wherein an average particle size is 1.0 μm or more and 3.0 μm or less.

### [A14]

An AFX-type zeolite, wherein
SAR (SiO₂/Al₂O₃ ratio) is 10 or more and 30 or less,
2θ=21.77°±0.15° in an XRD chart obtained by powder X-ray diffraction analysis corresponds to a strongest line,
an average particle size is 0.6 μm or more, and
a transition metal is supported.

### [A15]

A honeycomb stacked catalyst including the AFX-type zeolite according to [A14], and a honeycomb carrier.

### [A16]

A method for producing the AFX-type zeolite according to any of [A9] to [A14], the method comprising at least:
a step of preparing a mixture comprising at least:
   a silica and alumina source;
   an organic structure directing agent (OSDA) comprising a compound represented by the following formula (1) and/or a salt thereof:

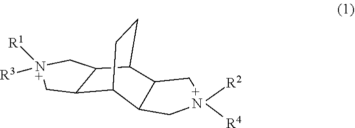

wherein R¹ to R⁴ are each independently an alkyl group;
an alkali metal hydroxide; and
water; and
a step of hydrothermally treating the mixture to synthesize the AFX-type zeolite.

### [A17]

A method for producing the AFX-type zeolite according to any of [A11] to [A14], the method comprising at least:
a step of preparing a mixture comprising at least:
   a silica and alumina source;
   an organic structure directing agent (OSDA) comprising a compound represented by the following formula (1) and/or a salt thereof:

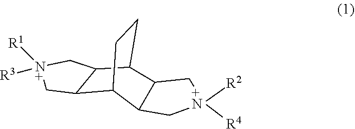

wherein R¹ to R⁴ are each independently an alkyl group;
an alkali metal hydroxide; and
water;
a step of hydrothermally treating the mixture to synthesize the AFX-type zeolite; and
a step of further calcining the AFX-type zeolite obtained, after the hydrothermally treating step.

### [A18]

A method for producing the AFX-type zeolite according to [A14], the method comprising at least:
a step of preparing a mixture comprising at least:
   a silica and alumina source;
   an organic structure directing agent (OSDA) comprising a compound represented by the following formula (1) and/or a salt thereof:

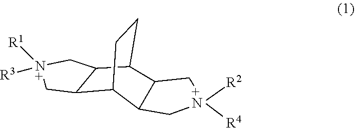

wherein R¹ to R⁴ are each independently an alkyl group;
an alkali metal hydroxide; and
water;
a step of hydrothermally treating the mixture to synthesize the AFX-type zeolite;
a step of further calcining the AFX-type zeolite obtained, after the hydrothermally treating step; and
a step of supporting a transition metal after the calcination step.

One aspect of the present invention provides various specific aspects shown below. Hereinafter, any aspect with respect to [B1] to [B8] is also referred to as any “specific aspect in a second group”.

### [B1]

A method for producing a compound represented by formula (1), or a salt thereof, comprising at least:
a step of providing a compound represented by formula (A) (step I);
a step of reacting the compound represented by the formula (A) with a hydrogen source by use of a catalyst, to thereby obtain a compound represented by formula (2) (step II); and
a step of N-alkylating the compound represented by the formula (2) with an alkylation reagent (step III);

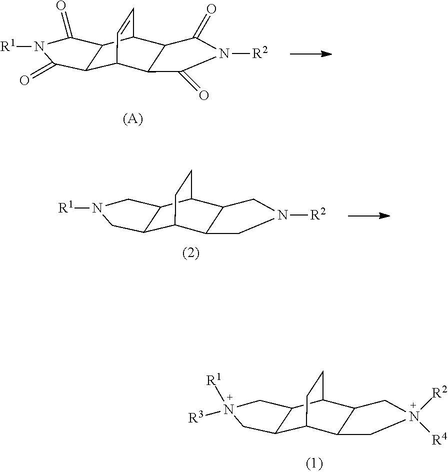

wherein R¹ to R⁴ are each independently an alkyl group.

### [B2]

The production method according to [B1], wherein the hydrogen source is molecular hydrogen.

### [B3]

The production method according to [B1] or [B2], wherein step II is performed under a wet process.

### [B4]

The production method according to any of [B1] to [B3], wherein the catalyst is a heterogeneous catalyst.

### [B5]

The production method according to any of [B1] to [B4], wherein the alkylation reagent is represented by R′—X, wherein R′ is an alkyl group and X is at least one leaving group selected from the group consisting a halogen atom and a sulfonyl group optionally having a substituent.

### [B6]

The production method according to [B5], wherein the alkylation reagent is an alkyl halide.

### [B7]

The production method according to [B6], wherein the alkylation reagent is an ethyl halide.

### [B8]

The production method according to any of [B1] to [B7], wherein R¹ and R² in the formula (A) and the formula (1) and (2) are each an ethyl group, and
R³ and R⁴ in the formula (1) are each an ethyl group.

One aspect of the present invention provides various specific aspects shown below. Hereinafter, any aspect with respect to [C1] to [C13] is also referred to as any “specific aspect in a third group”.

### [C1]

A method for producing an AFX-type zeolite, the method comprising at least:
a step of preparing a mixture comprising at least:
   a silica and alumina source;
   an organic structure directing agent (OSDA) comprising a compound represented by the following formula (1) and/or a salt thereof:

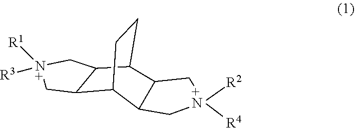

wherein R¹ to R⁴ are each independently an alkyl group;
an alkali metal hydroxide; and
water; and
a step of hydrothermally treating the mixture to synthesize an AFX-type zeolite.

### [C2]

The method for producing an AFX-type zeolite according to [C1], wherein R¹ to R⁴ in the formula (1) are the same alkyl groups.

### [C3]

The method for producing an AFX-type zeolite according to [C1] or [C2], wherein R¹ to R⁴ in the formula (1) are each an ethyl group.

### [C4]

The method for producing an AFX-type zeolite according to any of [C1] to [C3], wherein a silica/alumina ratio (SiO₂/Al₂O₃) in the mixture is 5 to 30.

### [C5]

The method for producing an AFX-type zeolite according to any of [C1] to [C4], wherein a compositional ratio except water in the AFX-type zeolite is represented by the following compositional ratio:
M_{a/b}Q_{c}Si_{48-d}Al_{d}O₉₆
wherein M represents a metal cation, a represents 1 to 10, b represents a valence of M, Q represents a cation derived from the compound represented by (1) and/or the salt thereof, c represents 0.5 to 2, and d represents 4 to 12.

### [C6]

The method for producing an AFX-type zeolite according to any of [C1] to [C5], wherein X-ray diffraction data of the AFX-type zeolite includes the following 2θ values) (°): 7.50±0.15, 8.71±0.15, 11.60 ±0.15, 13.01±0.15, 15.67±0.15, 17.46±0.15, 17.72±0.15, 19.93±0.15, 20.42±0.15, 21.84±0.15, 23.47±0.15, 26.19±0.15, 27.79±0.15, 30.67±0.15, 31.65±0.15, and 33.56±0.15.

### [C7]

The method for producing an AFX-type zeolite according to any of [C1] to [C6], wherein the mixture is prepared to further include a silica source (provided that any source corresponding to the silica and alumina source is excluded) in the step of preparing a mixture.

### [C8]

The method for producing an AFX-type zeolite according to any of [C1] to [C7], wherein the mixture is prepared to further include an alumina source (provided that any source corresponding to the silica and alumina source is excluded) in the step of preparing a mixture.

### [C9]

The method for producing an AFX-type zeolite according to any of [C1] to [C8], wherein the mixture is prepared to further include a seed crystal of aluminosilicate in the step of preparing a mixture.

### [C10]

The method for producing an AFX-type zeolite according to any of [C1] to [C9], including a step of further calcining the AFX-type zeolite obtained, after the hydrothermally treating step.

### [C11]

The method for producing an AFX-type zeolite according to [C10], wherein a compositional ratio except water in the AFX-type zeolite after calcination is represented by the following compositional ratio:
M_{a/b}H_{2c}Si_{48-d}Al_{d}O₉₆
wherein M represents a metal cation, a represents 1 to 10, b represents a valence of M, Q represents the compound represented by (1) and/or the salt thereof, and c represents 0.5 to 2, and d represents 4 to 12.

### [C12]

The method for producing an AFX-type zeolite according to any of [C1] to [C11], further including a step of exchanging an ion of the AFX-type zeolite obtained, with a NH⁴⁺ type ion and/or a H⁺ type ion.

### [C13]

The method for producing an AFX-type zeolite according to any of [C1] to [C12], the method comprising: a step of reacting N,N′-dialkylbicyclo[2.2.2]oct-7-ene-2,3:5,6-tetracarboxydiimide with a hydrogen source by use of a Pt—V/Z catalyst, to obtain N,N′-dialkylbicyclo[2.2.2]octane-2,3:5,6-dipyrrolidine; and
a step of N-alkylating the N,N′-dialkylbicyclo[2.2.2]octane-2,3:5,6-dipyrrolidine with an alkylation reagent, to thereby obtain the organic structure directing agent (OSDA) comprising a compound represented by formula (1) and/or a salt thereof.

One aspect of the present invention provides various specific aspects shown below. Hereinafter, any aspect with respect to [D1] is also referred to as any “specific aspect in a fourth group”.

### [D1]

An AFX-type zeolite having a macropore.

### [D2]

The AFX-type zeolite according to [D1], wherein
SAR (SiO₂/Al₂O₃ ratio) is 10 or more and 30 or less,
2θ=21.77°±0.15° in an XRD chart obtained by powder X-ray diffraction analysis corresponds to a strongest line, and
an average particle size is 0.6 μm or more.

### [D3]

The AFX-type zeolite according to [D1] or [D2], wherein X-ray diffraction data includes the following 2θ values) (°): 7.46±0.15, 8.69±0.15, 11.64±0.15, 12.93 ±0.15, 15.60±0.15, 17.43±0.15, 17.90±0.15, 19.81±0.15, 20.32±0.15, 21.77±0.15, 23.67 #0.15, 26.03±0.15, 28.05±0.15, 30.49±0.15, 31.50±0.15, and 33.71 ±0.15.

### [D4]

The AFX-type zeolite according to any of [D1] to [D3], wherein an average particle size is 1.0 μm or more and 3.0 μm or less.

### [D5]

The AFX-type zeolite according to [D1], wherein
SAR (SiO₂/Al₂O₃ ratio) is 10 or more and 30 or less,
2θ=21.77°±0.15° in an XRD chart obtained by powder X-ray diffraction analysis corresponds to a strongest line,
an average particle size is 0.6 μm or more, and
a transition metal is supported.

### [D6]

A honeycomb stacked catalyst including the AFX-type zeolite according to any one of [D1] to [D5], and a honeycomb carrier.

### [D7]

A honeycomb stacked catalyst, wherein a honeycomb carrier is coated with the AFX-type zeolite according to any one of [D1] to [D5].

### Advantageous Effects of Invention

A compound of one aspect of the present invention is useful as a compound (OSDA) serving as a starting material of a porous crystal material such as a zeolite.

According to a method for producing a compound according to one aspect of the present invention, a compound can be safely and easily provided which is useful as a compound (OSDA) serving as a starting material of a porous crystal material such as a zeolite.

According to a method for producing a zeolite according to one aspect of the present invention, an AFX-type zeolite can efficiently be produced.

According to a honeycomb stacked catalyst in which a honeycomb carrier is coated with an AFX-type zeolite, according to one aspect of the present invention, nitrogen oxide can be cleaned up with a reducing component at a high efficiency.

### BRIEF DESCRIPTION OF DRAWINGS

FIG. 1 A diagram illustrating ¹HNMR spectral data of a D₂O solution of N,N,N′,N′-tetraethylbicyclo[2.2.2]octane-2,3:5,6-dipyrrolidinium diiodide obtained in Example A1. In the Figure, * indicates a peak of an internal standard (4,4-dimethyl-4-silapentane-1-sulfonic acid).

FIG. 2 A diagram illustrating 13CNMR spectral data of a D₂O solution of N,N,N′,N′-tetraethylbicyclo[2.2.2]octane-2,3:5,6-dipyrrolidinium diiodide obtained in Example A1. In the Figure, * indicates a peak of an internal standard (4,4-dimethyl-4-silapentane-1-sulfonic acid).

FIG. 3A shows a diagram illustrating solid NMR spectral data of N,N,N′,N′-tetraethylbicyclo[2.2.2]octane-2,3:5,6-dipyrrolidinium.

FIG. 3B shows a solid NMR spectral data of an AFX-type zeolite obtained using N,N,N′,N′-tetraethylbicyclo[2.2.2]octane-2,3:5,6-dipyrrolidinium.

FIG. 3C shows a 13CNMR spectral data of a D₂O solution of an AFX-type zeolite obtained using N,N,N′,N′-tetraethylbicyclo[2.2.2]octane-2,3:5,6-dipyrrolidinium. * indicates a peak of an internal standard (4,4-dimethyl-4-silapentane-1-sulfonic acid).

FIG. 4 A diagram illustrating an XRD chart of an AFX-type zeolite of Example A2.

FIG. 5 A diagram illustrating an XRD chart of an AFX-type zeolite of Example A3.

FIG. 6 A diagram illustrating an XRD chart of an AFX-type zeolite of Comparative Example A1.

FIG. 7 A diagram illustrating an XRD chart of an AFX-type zeolite of Example A4.

FIG. 8 A diagram illustrating an XRD chart of an AFX-type zeolite of Example A5.

FIG. 9 A diagram illustrating an XRD chart of an AFX-type zeolite of Example A6.

FIG. 10 A diagram illustrating an XRD chart of an AFX-type zeolite of Comparative Example A2.

FIG. 11 A diagram illustrating the change in total integral intensity of XRD peaks before and after measurement of the hydrothermal durability of each AFX-type zeolite of Examples A4, A5 and A6, and Comparative Example A2.

FIG. 12 A diagram illustrating an SEM image of an AFX-type zeolite of Example A4.

FIG. 13 A diagram illustrating an SEM image of an AFX-type zeolite of Example A5.

FIG. 14 A diagram illustrating an SEM image of an AFX-type zeolite of Example A6.

FIG. 15 A diagram illustrating an SEM image of an AFX-type zeolite of Comparative Example A2.

FIG. 16 A diagram illustrating an XRD chart of a Cu-supported AFX-type zeolite of Example A7.

FIG. 17 A diagram illustrating an SEM image of a Cu-supported AFX-type zeolite of Example A7.

FIG. 18 A diagram illustrating an SEM image of a Cu-supported CHA-type zeolite of Comparative Example A3.

FIG. 19 A diagram illustrating an SEM image of a Cu-supported CHA-type zeolite of Comparative Example A4.

FIG. 20 A diagram illustrating 1HNMR spectral data of a D₂O solution of N,N,N′,N′-tetraethylbicyclo[2.2.2]octane-2,3:5,6-dipyrrolidinium diiodide obtained in Example B1. In the Figure, * indicates a peak of an internal standard (4,4-dimethyl-4-silapentane-1-sulfonic acid).

FIG. 21 A diagram illustrating 13CNMR spectral data of a D₂O solution of N,N,N′,N′-tetraethylbicyclo[2.2.2]octane-2,3:5,6-dipyrrolidinium diiodide obtained in Example B1. In the Figure, * indicates a peak of an internal standard (4,4-dimethyl-4-silapentane-1-sulfonic acid).

FIG. 22 A diagram illustrating XRD data of an AFX-type zeolite of Reference Example B1.

FIG. 23 A diagram illustrating ¹HNMR spectral data of a D₂O solution of N,N,N′,N′-tetraethylbicyclo[2.2.2]octane-2,3:5,6-dipyrrolidinium diiodide obtained in Reference Example B2. In the Figure, * indicates a peak of an internal standard (4,4-dimethyl-4-silapentane-1-sulfonic acid).

FIG. 24 A diagram illustrating ¹³CNMR spectral data of a D₂O solution of N,N,N′,N′-tetraethylbicyclo[2.2.2]octane-2,3:5,6-dipyrrolidinium diiodide obtained in Reference Example B2. In the Figure, * indicates a peak of an internal standard (4,4-dimethyl-4-silapentane-1-sulfonic acid).

FIG. 25 A diagram illustrating XRD data of an AFX-type zeolite of Reference Example B3.

FIG. 26 A diagram illustrating ¹HNMR spectral data of a D₂O solution of N,N,N′,N′-tetraethylbicyclo[2.2.2]octane-2,3:5,6-dipyrrolidinium diiodide obtained in Production Example C3. In the Figure, * indicates a peak of an internal standard (4,4-dimethyl-4-silapentane-1-sulfonic acid).

FIG. 27 A diagram illustrating ¹³CNMR spectral data of a D₂O solution of N,N,N′,N′-tetraethylbicyclo[2.2.2]octane-2,3:5,6-dipyrrolidinium diiodide obtained in Production Example C3. In the Figure, * indicates a peak of an internal standard (4,4-dimethyl-4-silapentane-1-sulfonic acid).

FIG. 28 A diagram illustrating XRD data of an AFX-type zeolite of Example C1.

FIG. 29 A diagram illustrating XRD data of a zeolite obtained in Comparative Example C1.

### DESCRIPTION OF EMBODIMENTS

Hereinafter, embodiments of the present invention are described in detail with reference to the drawings. The following embodiments are each one example (representative example) of aspects for carrying out the present invention, and the present invention is not limited thereto. In other words, the present invention can be arbitrarily modified and carried out without departing from the gist thereof. Herein, in a case where the term “to” is used for expression where numerical values or physical property values sandwich the term before and after the term, such values are used to be included. For example, the designation of a numerical value range of “1 to 100” encompasses both the lower limit value “1” and the upper limit value “100”. The same applies to designations of other numerical value ranges.

### (Compound)

A compound of the present embodiments corresponds to a compound represented by the following formula (1) or a salt thereof. Herein, the “compound or salt thereof” is also simply referred to as the “compound” encompassing the salt. The compound of the present embodiments is useful as OSDA. The compound of the present embodiments is industrially especially advantageous in that a compound which can be simply and stably synthesized can be used as a starting material without use of any reducing agent reagent whose handling and reaction control are difficult, such as LiAlH₄. In a case where the compound of the present embodiments is used to produce an AFX-type zeolite, such an AFX-type zeolite can be obtained in the form of a single phase.

Herein, a compound represented by the following formula (1) is also referred to as “N,N,N′,N′-tetraalkylbicyclo[2.2.2]octane-2,3:5,6-dipyrrolidinium”.

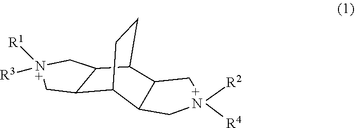

In the formula (1), R¹ to R⁴ are each independently an alkyl group. R¹ to R⁴ are preferably the same alkyl groups.

Such an alkyl group can be suitably, for example, a straight or branched alkyl group having 1 to 4 carbon atoms, and specific examples thereof include a methyl group, an ethyl group, a n-propyl group, an isopropyl group, a n-butyl group, an isobutyl group, a sec-butyl group, and a tert-butyl group.

Among such alkyl groups, an alkyl group having 1 to 3 carbon atoms is preferable, and an alkyl group having 1 to 2 carbon atoms is more preferable. Specifically, such an alkyl group is preferably a methyl group, an ethyl group, an n-propyl group, or an isopropyl group, more preferably a methyl group or an ethyl group, further preferably an ethyl group.

Specific examples of the compound represented by the formula (1) can include the following compounds.

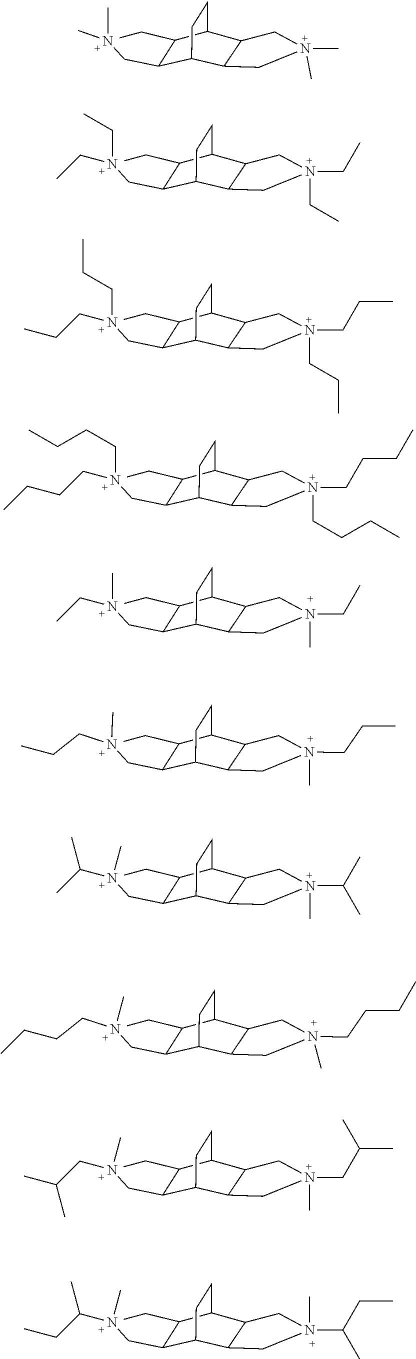

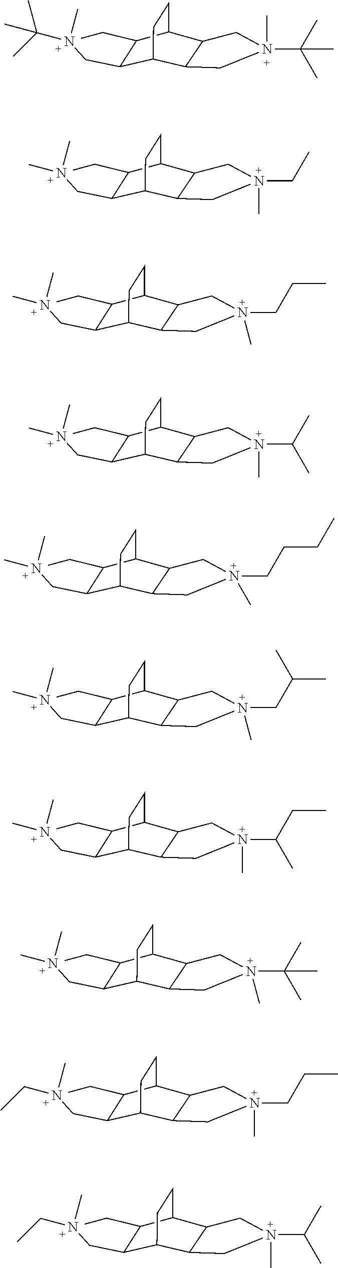

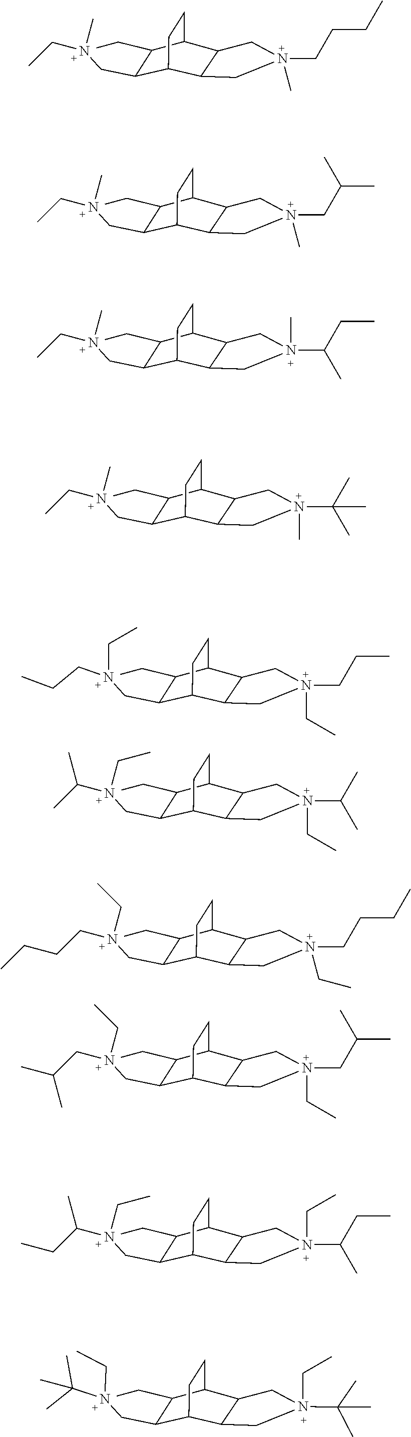

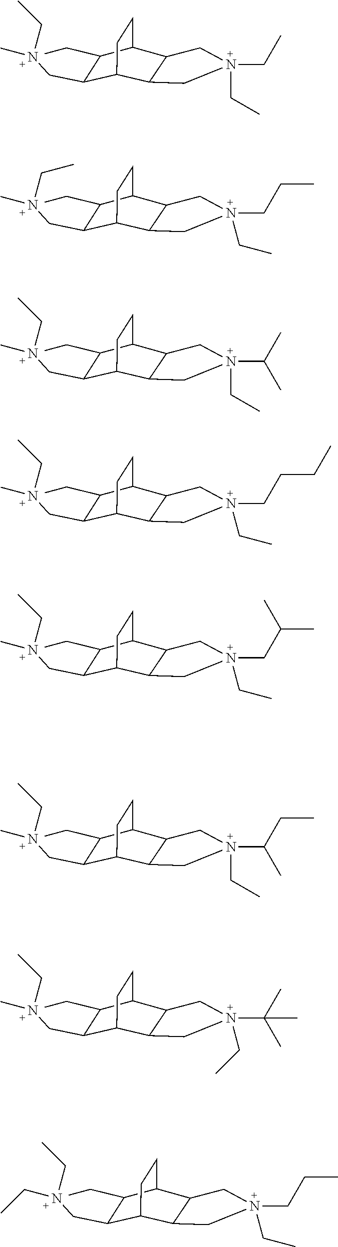

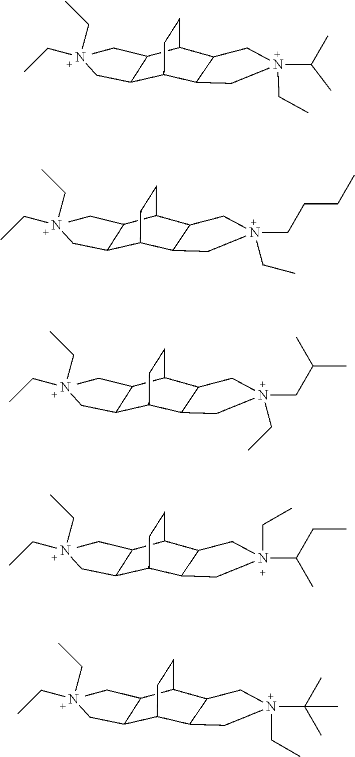

The compound of the present embodiments also encompasses a salt thereof, as described above. A counter anion which is taken together with an ammonium cation of the compound of the present embodiments to form a salt is not particularly limited, and may be an inorganic anion or an organic anion. Examples of the counter anion in formation of a salt of the present embodiments include a hydroxide ion, a nitrate ion, a sulfate ion, a carbonate ion, a hydrogen carbonate ion, a halide ion (fluorine, chlorine, bromine, iodine), a formate ion, an acetate ion, a citrate ion, a tartrate ion, an oxalate ion, a fumarate ion, and an anion of a saturated or unsaturated linear fatty acid having 3 to 20 carbon atoms. In particular, a hydroxide ion or a halide ion is preferable. In other words, the salt of the compound of the present embodiments is preferably hydroxide or halide. The salt of the compound of the present embodiments may be a mixture of two or more different kinds thereof.

### (Production Method)

The compound represented by the formula (1) of the present embodiments can be produced according to a known synthesis route, and the production method thereof is not particularly limited. One preferable production method is, for example, a production method including a step of N-alkylating the following compound represented by formula (2) with an alkylation reagent. The following compound represented by formula (2) is herein also referred to as “N,N′-dialkylbicyclo[2.2.2]octane-2,3:5,6-dipyrrolidine”. A particularly suitable production method of the present embodiments can be represented by the following scheme.

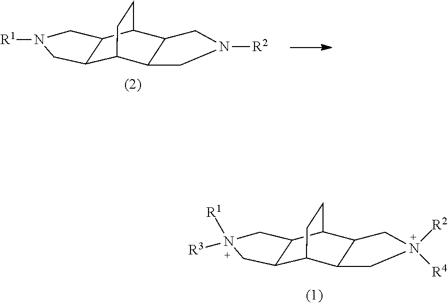

R¹ to R⁴ in the scheme are each independently an alkyl group. R¹ and R² in the compound represented by the formula (2) have the same meanings as R¹ and R² in the formula (1), and preferable substituents can also include the same groups as in R¹ and R² in the formula (1).

The alkylation reagent is not particularly limited as long as nitrogen in the compound represented by the formula (2) is alkylated, and examples thereof can include an alkylation reagent represented by R′—X. R′ is an alkyl group, and X is a leaving group. Examples of the leaving group suitably include halogen atoms such as a chlorine atom, a bromine atom, and an iodine atom; and sulfonyl groups such as methylsulfonyl, trifluoromethylsulfonyl, and p-toluenesulfonyl groups.

The alkylation reagent is preferably an alkyl halide, more preferably a methyl halide or an ethyl halide.

N,N,N′,N′-tetraethylbicyclo[2.2.2]oct-7-ene-2,3:5,6-dipyrrolidinium hydroxide has been used in synthesis of an AFX-type zeolite in a conventional art (see Japanese Patent Laid-Open No. 2016-169139 (Patent Literature 2)). In synthesis of the hydroxide, a solution of the hydroxide is obtained by exchanging an ion of N,N,N′,N′-tetraethylbicyclo[2.2.2]oct-7-ene-2,3:5,6-dipyrrolidiniumiodide obtained as a solid powder with a hydroxide-type negative ion-exchange resin, and concentrating the resultant.

On the other hand, the compound (salt) of the present embodiments, which is in the form of a salt obtained by alkylation as described above, namely, a salt where X is a counter anion, can be used as it is, for synthesis of a zeolite. Accordingly, in this case, the labor for preparation of the hydroxide can be avoided, and a zeolite can be efficiently produced. For example, in the case of use as the hydroxide, for example, ion-exchange with a hydroxide-type negative ion-exchange resin and concentration may be performed similarly to those conventionally performed.

The amount of the alkylation reagent used may be appropriately set in consideration of synthesis efficiency, purity, and the like and is not particularly limited, the target thereof is usually 2 equivalents or more relative to the molar number of the compound represented by the formula (2), preferably 2 to 50 equivalents, more preferably 2 to 10 equivalents.

The reaction in the present embodiments may be performed in the presence of a solvent, namely, under a wet process.

The solvent is not particularly limited as long as the solvent can dissolve the compound represented by the formula (2), and may be appropriately selected depending on, for example, the reaction temperature and the reactants.

Examples of the solvent include water; aromatic hydrocarbon-based solvents such as benzene and toluene; amide-based solvents such as acetonitrile, N,N-dimethylacetamide, and N,N-dimethylformamide; ether-based solvents such as tetrahydrofuran (hereinafter, also designated as “THF”), diethyl ether, and 1,2-dimethoxyethane; alcohol-based solvents such as methanol, ethanol, and isopropanol; and halogen-based solvents such as dichloromethane, dichloroethane, and chloroform. Such solvents can be used singly or in any combination of two or more kinds thereof at any ratio.

Among such solvents, an alcohol-based solvent is preferable.

Use of the solvent and the amount thereof used may be appropriately set in consideration of other reaction conditions, and are not particularly limited, and the concentration of the compound represented by the formula (2) in the reaction mixture is preferably 0.001 to 10 mol/L, more preferably 0.01 to 5 mol/L, further preferably 0.01 to 3 mol/L.

The reaction temperature is not particularly limited, and may be appropriately adjusted depending on, for example, the type of the solvent. The reaction temperature is usually in the range from 20 to 200° C., preferably in the range from 50 to 150° C., more preferably in the range from 50 to 120° C. The reaction may also be performed at a temperature at which the solvent is refluxed.

The reaction time may be appropriately adjusted by monitoring progress of the reaction by use of, for example, GC-MS, and is usually 1 minute to 100 hours, preferably 0.5 hours to 70 hours, more preferably 1 hour to 60 hours.

In a case where the solvent is used in the reaction, the mixture after completion of the reaction, which is in the form of a reaction solution obtained, may be, if necessary, concentrated and thereafter the residue may be used as a raw material as it is, or the reaction mixture may be appropriately subjected to a post-treatment to obtain the compound represented by the formula (1). Specific methods for the post-treatment can include known purification methods such as water washing, filtration, drying, extraction, distillation, and chromatography. Such purification methods may be performed in combinations of two or more kinds thereof.

The salt may also be obtained by performing adjustment of the counter anion by use of an ion-exchange resin or the like, as the post-treatment. Specifically, a desired salt can be obtained by appropriately dissolving a compound obtained after the step of N-alkylating the compound represented by the formula (2) with an alkylation reagent, in the solvent, and contacting the resultant with an ion-exchange resin.

The compound represented by the formula (2) can be produced by a known synthesis route, and the production method thereof is not particularly limited. The compound represented by the formula (2) can be produced by hydrogenating N,N′-dialkylbicyclo[2.2.2]oct-7-ene-2,3:5,6-dipyrrolidine which can be synthesized according to Patent Literature 2, as represented by, for example, the following scheme.

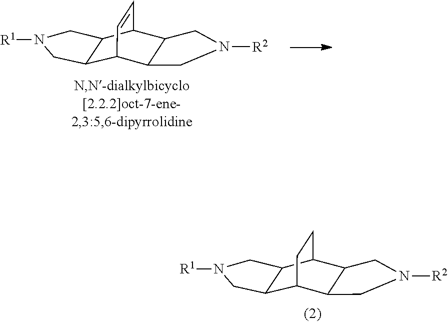

R¹ and R² in the scheme are each independently an alkyl group. R¹ and R² in N,N′-dialkylbicyclo[2.2.2]oct-7-ene-2,3:5,6-dipyrrolidine have the same meanings as R¹ and R² in the formula (1), and preferable substituents can also include the same groups as in R¹ and R² in the formula (1).

The compound represented by the formula (2) is specifically produced by reacting N,N′-dialkylbicyclo[2.2.2]oct-7-ene-2,3:5,6-dipyrrolidine with a hydrogen source in the presence or absence of a catalyst.

The hydrogen source used in the production method can be appropriately selected from those capable of hydrogenating N,N′-dialkylbicyclo[2.2.2]oct-7-ene-2,3:5,6-dipyrrolidine. Specific examples include molecular hydrogen such as a hydrogen gas; and hydrogen donors such as ammonium formate, sodium formate, and hydrazine, but not particularly limited thereto. Among such hydrogen sources, molecular hydrogen is preferable. In a case where molecular hydrogen is used in the production method, the hydrogen pressure in the reactor is usually 0.1 to 10 MPa, preferably 0.1 to 5 MPa, more preferably 0.1 to 1.0 MPa.

The reaction in the present embodiments may be performed in the presence of a solvent, namely, under a wet process. Examples of the solvent include water; aromatic hydrocarbon-based solvents such as benzene and toluene; amide-based solvents such as acetonitrile, N,N-dimethylacetamide, and N,N-dimethylformamide; ether-based solvents such as THE, diethyl ether, and 1,2-dimethoxyethane; alcohol-based solvents such as methanol, ethanol, and isopropanol; and halogen-based solvents such as dichloromethane, dichloroethane, and chloroform. Such solvents can be used singly or in any combination of two or more kinds thereof at any ratio. Among such solvents, an alcohol-based solvent is preferable.

Use of the solvent and the amount thereof used may be appropriately set in consideration of other reaction conditions, and are not particularly limited, and the concentration of N,N′-dialkylbicyclo[2.2.2]oct-7-ene-2,3:5,6-dipyrrolidine in the reaction mixture is preferably 0.001 to 10 mol/L, more preferably 0.01 to 5 mol/L, further preferably 0.01 to 3 mol/L.

The reaction temperature is not particularly limited, and may be appropriately adjusted depending on, for example, the type of the solvent. The reaction temperature is usually in the range from 20 to 200° C., preferably in the range from 20 to 150° C., more preferably in the range from 30 to 120° C. The reaction may also be performed at a temperature at which the solvent is refluxed.

The reaction time may be appropriately adjusted by monitoring progress of the reaction by use of, for example, GC-MS, and is usually 1 minute to 1000 hours, preferably 0.5 hours to 300 hours, more preferably 1 hour to 200 hours.

In a case where the solvent is used in the reaction, the mixture after completion of the reaction, which is in the form of a reaction solution obtained, may be, if necessary, concentrated and thereafter the residue may be used as a raw material as it is, or the reaction mixture may be appropriately subjected to a post-treatment to obtain the compound represented by the formula (2). Specific methods for the post-treatment can include known purification methods such as water washing, filtration, drying, extraction, distillation, and chromatography. Such purification methods may be performed in combinations of two or more kinds thereof.

### (Structure Directing Agent for Zeolite Synthesis)

The compound and the salt thereof, of the present embodiments, can be each used as a structure directing agent (OSDA; Organic Structure Directing Agents) in zeolite production. In other words, one of the present embodiments relates to a structure directing agent for zeolite synthesis, including a compound represented by formula (1) and/or a salt thereof.

### <AFX-Type Zeolite and Production Method Thereof>

A method for producing an AFX-type zeolite of the present embodiments includes at least a step of preparing a mixture including at least a silica and alumina source, an organic structure directing agent (OSDA) including a compound represented by the following (1) and/or a salt thereof, an alkali metal hydroxide, and water, and a step of hydrothermally treating the mixture to synthesize an AFX-type zeolite:

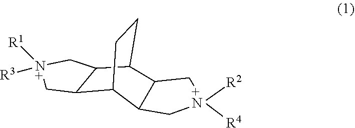

wherein R¹ to R⁴ are each independently an alkyl group.

The compound represented by (1) and/or the salt thereof are/is used in the organic structure directing agent (OSDA) in the production method of the present embodiments. The compound represented by (1) and the salt thereof are high in availability. In a case where the compound represented by (1) and/or the salt thereof are/is used in the organic structure directing agent (OSDA), an AFX-type zeolite can be obtained in the form of a single phase without being miscible with other phases. As described above, the production method of the present embodiments can efficiently produce an AFX-type zeolite.

An AFX-type zeolite of the present embodiments is an aluminosilicate to which a three-letter code “AFX” is given by International Zeolite Association Structure Commission (IZA-SC).

### (Silica and Alumina Source)

The silica and alumina source used as a starting material is any known one without any limitation as long as it includes at least an aluminosilicate (Si—Al element source) where the silica/alumina ratio (SiO₂/Al₂O₃, hereinafter, sometimes referred to as “SAR”) is 2 or more and less than 50. The type is not particularly limited. The aluminosilicate here means one having a structure where some silicon atoms in silicate are each replaced with an aluminum atom. The crystal form of such a silica/alumina source is not particularly limited, and may be amorphous or may have a zeolite structure like FAU. The silica/alumina ratio is preferably 5 or more and less than 40, more preferably 10 or more and 30 or less. The silica/alumina ratio herein means a value determined from fluorescent X-ray analysis. Specifically, SAR is calculated from the results of respective percentages by mass of Al₂O₃ and SiO₂, in which the results are obtained by molding about 5 g of a specimen under pressure at 20 t to provide a sample, and subjecting the sample to measurement with Axios (Spectris).

The above-mentioned Si—Al element source can be used singly, as the silica and alumina source used in the present embodiments, but a Si element source (provided that any source corresponding to the Si—Al element source is excluded) and an Al element source (provided that any source corresponding to the Si—Al element source is excluded) may be used in combination, or a mixture of a Si element source and an Al element source can also be used as the silica and alumina source. For example, the silica and alumina source may be an aspect where the Si—Al element source is used further in combination with a Si element source (provided that any source corresponding to the Si—Al element source is excluded) and/or an Al element source (provided that any source corresponding to the Si—Al element source is excluded). In particular, the Si—Al element source is preferably used singly.

Examples of the Si element source include precipitated silica, colloidal silica, fumed silica, silica gel, sodium silicates (for example, sodium metasilicate, sodium orthosilicate, and silicate soda Nos. 1, 2, 3 and 4), and alkoxysilanes such as tetraethoxysilane (TEOS) and trimethylethoxysilane (TMEOS), but not particularly limited thereto. It is noted that any aluminosilicate where the SAR is 2 or more and less than 20 herein corresponds to the above-mentioned Si—Al element source and is not encompassed in the Si element source.

The Si element source can be herein used singly or in any combination of two or more kinds thereof at any ratio.

Examples of the Al element source include aluminum hydroxide, sodium aluminate, aluminum hydroxide oxide, and aluminum oxide, but not particularly limited thereto. It is noted that any aluminosilicate where the SAR is 2 or more and less than 50 herein corresponds to the above-mentioned Si—Al element source and is not encompassed in the Al element source.

The Al element source can be used singly or in any combination of two or more kinds thereof at any ratio.

The SAR in the AFX-type zeolite of the present embodiments is also preferably 2 or more and less than 50, more preferably 5 or more and less than 40, further preferably 10 or more and 30 or less. The SAR in the AFX-type zeolite can be adjusted to the range by using an aluminosilicate (Si—Al element source) where the SAR is 2 or more and less than 50 in production of the AFX-type zeolite, as described above.

### (Alkali Metal Hydroxide)

Examples of the alkali metal source include alkali metal hydroxides such as LiOH, NaOH, KOH, CsOH, and RbOH, aluminate of such an alkali metal, and any alkali component included in the above-mentioned Si—Al element source and Si element source. In particular, NaOH or KOH is suitably used. The alkali metal in the mixture can also function as an inorganic structure directing agent, resulting in a tendency to easily obtain an aluminosilicate excellent in crystallinity.

The alkali metal source can be used singly or in any combination of two or more kinds thereof at any ratio.

### (Water)

The water here used may be any of, for example, tap water, RO water, deionized water, distilled water, industrial water, pure water, and ultrapure water, depending on desired performance. The method for compounding the water with the mixture may be made by compounding the water separately from each of the above-mentioned components, or may be made by mixing the water with each of the components in advance and compounding an aqueous solution or a dispersion liquid of each of the components.

In a step of preparing a mixture, a mixture (slurry) including the above-mentioned silica and alumina source, an organic structure directing agent (OSDA) including the compound represented by the formula (1) and/or the salt thereof, an alkali metal hydroxide, and water is prepared. Wet mixing can be here made by use of, if necessary, a known mixer or stirrer, such as a ball mill, a bead mill, a medium stirring mill, or a homogenizer. In a case where stirring is performed, the stirring is preferably performed usually at a number of rotations of about 30 to 2000 rpm, more preferably 50 to 1000 rpm.

The content of the water in the mixture can be appropriately set in consideration of, for example, reactivity and handleability and is not particularly limited, and the water/silica ratio (H₂O/SiO₂ molar ratio) in the mixture is usually 5 or more and 100 or less, preferably 6 or more and 50 or less, more preferably 7 or more and 40 or less. The water/silica ratio is within the preferable range, to thereby facilitate stirring in mixture preparation or in crystallization by hydrothermal synthesis, thereby not only enhancing handleability, but also inhibiting crystals of a side product and impurities from being produced, resulting in a tendency to easily provide a high yield. The method for compounding the water with the mixture may be made by compounding the water separately from each of the above-mentioned components, or may be made by mixing the water with each of the components in advance and compounding an aqueous solution or a dispersion liquid of each of the components.

The silica/alumina ratio (SiO₂/Al₂O₃) in the mixture can also be appropriately set, is not particularly limited, and is usually 5 or more and 50 or less, preferably 7 or more and less than 45, further preferably 10 or more and 30 or less. The silica/alumina ratio is within the preferable range, to thereby provide a dense crystal also sufficiently having an effective cation site for a catalyst reaction, resulting in a tendency to provide an aluminosilicate excellent in thermal durability under a high temperature environment or after exposure to a high temperature.

In this regard, the hydroxide ion/silica ratio (molar ratio of OH⁻/SiO₂) in the mixture can also be appropriately set, is not particularly limited, and is usually 0.10 or more and 0.90 or less, preferably 0.15 or more and 0.50 or less, further preferably 0.20 or more and 0.40 or less. The hydroxide ion/silica ratio is within the preferable range, to thereby allow crystallization to easily progress, resulting in a tendency to provide an aluminosilicate excellent in thermal durability under a high temperature environment or after exposure to a high temperature.

The content of the alkali metal in the mixture can also be appropriately set and is not particularly limited, and the molar ratio in terms of alkali metal (M) oxide, namely, the alkali metal oxide/silica ratio (M₂O/SiO₂ molar ratio) is usually 0.01 or more and 0.50 or less, preferably 0.05 or more and 0.30 or less. The alkali metal oxide/silica ratio is within the preferable range, to thereby not only promote crystallization by mineralizing action, but also inhibit crystals of a side product and impurities from being produced, resulting in a tendency to easily provide a high yield.

In this regard, the organic structure directing agent/silica ratio (molar ratio of organic structure directing agent/SiO₂) in the mixture can also be appropriately set, is not particularly limited, and is usually 0.05 or more and 0.40 or less, preferably 0.07 or more and 0.30 or less, further preferably 0.09 or more and 0.25 or less. The organic structure directing agent/silica ratio is within the preferable range, to thereby allow crystallization to easily progress, resulting in a tendency to provide an aluminosilicate excellent in thermal durability under a high temperature environment or after exposure to a high temperature, at low cost.

The above-mentioned mixture may contain a specified anion from the viewpoints of promotion of crystallization and control of the crystal grain size. For example, as in Patent Literature 2, in a case where a mixture is produced from OSDA including only a hydroxide of the compound represented by the formula (1), without addition of any specified anion, an AFX-type zeolite relatively small in crystal grain size is obtained. In this regard, in a case where a halide ion is included in the mixture, an AFX-type zeolite relatively large in crystal grain size can be obtained. The halide ion may be any of fluoride, chloride, bromide, and iodide ions. The method for allowing the halide ion to be included in the mixture is not particularly limited, and the halide ion may be added as a counter ion of OSDA, may be added as a counter ion of an alkali metal, or may be added as a free acid.

The above-mentioned mixture may further contain a seed crystal of an aluminosilicate having a desired skeleton structure, from the viewpoint of, for example, promotion of crystallization. The seed crystal is compounded to thereby promote crystallization of a desired skeleton structure, resulting in a tendency to provide a high-quality aluminosilicate. The seed crystal here used is not particularly limited as long as it has a desired skeleton structure. The seed crystal here used can be, for example, a seed crystal of an aluminosilicate having at least one skeleton structure of CHA, AEI, ERI, and AFX. The silica/alumina ratio in the seed crystal is any value and is preferably a value identical to or comparable with the silica/alumina ratio in the mixture, and the silica/alumina ratio in the seed crystal is preferably 5 or more and 50 or less, more preferably 8 or more and less than 40, further preferably 10 or more and less than 30, from such a viewpoint.

The seed crystal here used can be any of not only an aluminosilicate separately synthesized, but also a commercially available aluminosilicate. Of course, a natural aluminosilicate can also be used, or an aluminosilicate synthesized by the present invention can also be used as the seed crystal. The cation type of the seed crystal is not particularly limited, and can be, for example, a sodium type, a potassium type, an ammonium type, or a proton type.

The particle size (D₅₀) of the seed crystal here used is not particularly limited, and is desirably relatively small from the viewpoint of promotion of crystallization of a desired crystal structure, and is usually 0.5 nm or more and 5 μm or less, preferably 1 nm or more and 3 μm or less, more preferably 2 nm or more and 1 μm or less. The amount of the seed crystal compounded can be appropriately set depending on desired crystallinity, is not particularly limited, and is preferably 0.05 to 30% by mass, more preferably 0.1 to 20% by mass, further preferably 0.5 to 10% by mass, based on the mass of SiO₂ in the mixture.

In a step of hydrothermally treating the mixture, the mixture is heated in a reaction container and thus subjected to hydrothermal synthesis, to thereby an aluminosilicate (AFX-type zeolite) crystallized.

The reaction container used in the hydrothermal synthesis can be appropriately a known reaction container as long as such a known reaction container is a sealed pressure-resistant container which can be used in the hydrothermal synthesis, and the type thereof is not particularly limited. For example, a sealed heat-resistant and pressure-resistant container such as an autoclave equipped with a stirring apparatus, a heat source, a pressure gauge, and a safety valve is preferably used. Herein, crystallization of an aluminosilicate may be performed in a state where the above-mentioned mixture (starting material composition) is left to stand still, or may be performed in a state where the above-mentioned mixture (starting material composition) is stirred and mixed, from the viewpoint of an enhancement in uniformity of an aluminosilicate obtained. Such crystallization is preferably performed usually at a number of rotations of about 30 to 2000 rpm, more preferably 50 to 1000 rpm. Such stirring may be intermittently performed for the purpose of, for example, control of the crystal grain size.

The treatment temperature (reaction temperature) of the hydrothermal synthesis is not particularly limited, and is usually 100° C. or more and 200° C. or less, preferably 120° C. or more and 190° C. or less, more preferably 150° C. or more and 180° C. or less, from the viewpoint of, for example, crystallinity and economic performance of an aluminosilicate obtained.

The treatment time (reaction time) of the hydrothermal synthesis is not particularly limited as long as a sufficient time can be taken for crystallization, and is usually 1 hour or more and 20 days or less, preferably 4 hours or more and 15 days or less, more preferably 12 hours or more and 10 days or less, from the viewpoint of, for example, crystallinity and economic performance of an aluminosilicate obtained.

The treatment pressure of the hydrothermal synthesis is not particularly limited, and is sufficiently an autogenic pressure generated in heating of the mixture loaded into the reaction container, to the temperature range. An inert gas such as nitrogen or argon may be, if necessary, introduced into the container.

Such a hydrothermal treatment can be performed to provide an aluminosilicate crystallized. For example, a solid-liquid separation treatment, a water washing treatment, or, for example, a drying treatment for removal of moisture in air at a temperature of about 50 to 150° C. may be, if necessary, performed according to an ordinary method.

The compositional ratio except water in the AFX-type zeolite obtained in the hydrothermally treating step is preferably represented by the following compositional ratio:
M_{a/b}Q_{c}Si_{48-d}Al_{d}O₉₆
wherein M represents a metal cation, a represents 1 to 10, b represents a valence of M, Q represents a cation derived from the compound represented by (1) and/or the salt thereof, and c represents 0.5 to 2, and d represents 4 to 12.

The AFX-type zeolite having the compositional ratio is also referred to as “AFX-type zeolite before calcination”. The AFX-type zeolite having the compositional ratio corresponds to one of the present embodiments. The X-ray diffraction data of the AFX-type zeolite preferably includes the following 2θ values) (°): 7.50±0.15, 8.71±0.15, 11.60±0.15, 13.01±0.15, 15.67±0.15, 17.46±0.15, 17.72±0.15, 19.93±0.15, 20.42±0.15, 21.84±0.15, 23.47±0.15, 26.19±0.15, 27.79±0.15, 30.67±0.15, 31.65±0.15, and 33.56±0.15.

M in the compositional formula is usually a Na cation. The compositional ratio represents a compositional ratio per unit cell of the AFX-type zeolite.

The AFX-type zeolite thus obtained may include a structure directing agent, an alkali metal, and/or the like in a pore or the like. Thus, a removal step of removing such structure directing agent, alkali metal, and/or the like is, if necessary, preferably performed. Removal of such organic structure directing agent, alkali metal, and/or the like can be performed according to an ordinary method, and such a method is not particularly limited. For example, a liquid phase treatment using an acidic aqueous solution, a liquid phase treatment using an aqueous solution containing an ammonium ion, a liquid phase treatment using a chemical liquid including a decomposed component of the organic structure directing agent, an exchange treatment using a resin or the like, or a calcination treatment can be performed. Such treatments can be performed in the form of any combination thereof. In particular, removal of such organic structure directing agent, alkali metal, and/or the like is preferably performed by using a calcination treatment from the viewpoint of, for example, production efficiency.

The treatment temperature (calcination temperature) in the calcination treatment can be appropriately set depending on, for example, the starting material used, is not particularly limited, and is usually 300° C. or more and 1000° C. or less, preferably 400° C. or more and 900° C. or less, more preferably 430° C. or more and 800° C. or less, further preferably 480° C. or more and 750° C. or less, from the viewpoint that, for example, not only crystallinity is maintained, but also the remaining percentage of such structure directing agent, alkali metal, and/or the like is reduced. The calcination treatment is preferably performed in an oxygen-containing atmosphere, and may be performed, for example, in an air atmosphere.

The treatment time (calcination time) in the calcination treatment can be appropriately set depending on, for example, the treatment temperature and economic performance, is not particularly limited, and is usually 0.5 hours or more and 72 hours or less, preferably 1 hour or more and 48 hours or less, more preferably 3 hours or more and 40 hours or less.

The compositional ratio except water in the AFX-type zeolite after calcination is preferably represented by the following compositional ratio:
M_{a/b}Q_{2c}Si_{48-d}Al_{d}O₉₆
wherein M represents a metal cation, a represents 1 to 10, b represents a valence of M, Q represents the compound represented by (1) and/or the salt thereof, and c represents 0.5 to 2, and d represents 4 to 12. The AFX-type zeolite having the compositional ratio corresponds to one of the present embodiments.

One AFX-type zeolite of the present embodiments is an AFX-type zeolite in which the SAR (SiO₂/Al₂O₃ ratio) is 10 or more and 30 or less, 2θ=21.77°±0.15° in an XRD chart obtained by powder X-ray diffraction analysis corresponds to a strongest line, and the average particle size is 0.6 μm or more. This AFX-type zeolite can be obtained by, for example, performing a calcination treatment of the above-mentioned AFX-type zeolite before calcination. The X-ray diffraction data of the AFX-type zeolite preferably includes the following 2θ values) (°): 7.46±0.15, 8.69±0.15, 11.64±0.15, 12.93±0.15, 15.60±0.15, 17.43±0.15, 17.90±0.15, 19.81±0.15, 20.32±0.15, 21.77±0.15, 23.67±0.15, 26.03±0.15, 28.05±0.15, 30.49±0.15, 31.50±0.15, 33.71±0.15.

The average particle size of the AFX-type zeolite of the present embodiments is preferably 0.01 μm to 20 μm, more preferably 0.6 to 6.0 μm, further preferably 0.7 μm to 4.0 μm, particularly preferably 1.0 μm to 3.5 μm.

M in the compositional formula is usually a Na cation. The compositional ratio represents a compositional ratio per unit cell of the AFX-type zeolite.

### <Ion-Exchange>

The aluminosilicate after crystallization may have a metal ion such as an alkali metal ion on the ion-exchange site. An ion-exchange step of performing ion-exchange can be here performed depending on desired performance. In the ion-exchange step, ion-exchange with a non-metal cation such as an ammonium ion (NH₄⁺) or proton (H⁺) can be performed according to an ordinary method. For example, ion-exchange with an ammonium-type ion can be made by subjecting an aluminosilicate to a liquid phase treatment using an aqueous solution containing an ammonium ion, such as an aqueous ammonium nitrate solution or an aqueous ammonium chloride solution. Alternatively, ion-exchange with a proton-type can be made by subjecting an aluminosilicate to ion-exchange with ammonia and then to a calcination treatment. In the production method, an ammonium ion (NH₄⁺) type is preferable from the viewpoint that a treatment liquid neutralized in a P supporting treatment is used to omit a calcination treatment and a high-temperature drying treatment. An aluminosilicate thus obtained can also be, if necessary, subjected to a treatment such as a reduction in amount of acid. Such a treatment for a reduction in amount of acid can be performed by, for example, silylation, a steam treatment, or a dicarboxylic acid treatment. Such a treatment for a reduction in amount of acid, and the change in compositional ratio may be each performed according to an ordinary method.

### <Supporting of Transition Metal>

A transition metal-supported zeolite can also be obtained by, if necessary, supporting a transition metal on the above-mentioned aluminosilicate (which is an aluminosilicate with no transition metal supported). Such a transition metal supporting treatment may be performed according to an ordinary method. The transition metal can be thus supported to thereby allow for the function as a catalyst in various applications. Examples of the transition metal here supported include copper (Cu), iron (Fe), and tungsten (W), but not particularly limited thereto.

Such a transition metal supporting treatment may be performed according to an ordinary method. For example, the treatment may be performed by contacting the above-mentioned aluminosilicate with, for example, a single transition metal or a compound thereof, or a transition metal ion. The method for supporting the transition metal may be any method involving retaining the transition metal on at least any of an ion-exchange site or a pore of the aluminosilicate. The transition metal can be supplied in the form of an inorganic acid salt of the transition metal, for example, sulfate, nitrate, acetate, chloride, oxide, composite oxide, and a complex salt of the transition metal. In particular, in a case where a P supporting treatment is performed, a treatment liquid neutralized in the treatment is used and thus the transition metal is preferably supplied in the form of an inorganic salt of a strong acid, such as sulfate or nitrate. Examples of a specific method include an ion-exchange method, an evaporation-to-dryness method, a precipitation supporting method, a physical mixing method, a skeleton substitution method, and an impregnation support method, but not particularly limited thereto. After the transition metal supporting treatment, for example, a solid-liquid separation treatment, a water washing treatment, or a drying treatment for removal of moisture, for example, in air at a temperature of about 50 to 150° C. can be, if necessary, performed according to an ordinary method.

A platinum group element (PGM: Platinum Group Metal) such as platinum, palladium, rhodium, or iridium may be, if necessary, supported on the aluminosilicate. A known procedure can be applied to the method for supporting a noble metal element or a platinum group element, and is not particularly limited. For example, a noble metal element or a platinum group element can be supported by preparing a solution of a salt including the noble metal element or the platinum group element, impregnating the aluminosilicate with such a salt-containing solution, and thereafter calcining the resultant. The salt-containing solution is not particularly limited, and is preferably, for example, an aqueous nitrate solution, a dinitrodiammine nitrate solution, or an aqueous chloride solution. The calcination treatment is also not particularly limited, and is preferably made at 350° C. to 1000° C. for about 1 to 12 hours. It is here preferable to perform a drying treatment including drying under reduced pressure by use of a vacuum dryer or the like at about 50° C. to 180° C. for about 1 to 48 hours, before calcination at a high temperature.

Next, such a zeolite with no transition metal supported or such a transition metal-supported zeolite, thus provided, is described. Such a zeolite with no transition metal supported or such a transition metal-supported zeolite corresponds to a crystalline aluminosilicate classified by an AFX structure code in various structure codes in IZA. An AFX-type zeolite has a structure which has aluminum (Al) and silicon (Si) as main skeleton metal atoms and which is made of a network of such atoms and oxygen (O). The structure is characterized according to X-ray diffraction data.

The particle size of the zeolite with no transition metal supported or the transition metal-supported zeolite can vary depending on synthesis conditions and the like and thus is not particularly limited, and the average particle size (D₅₀) thereof is preferably 0.01 μm to 20 μm, more preferably 0.6 to 6.0 μm, further preferably 0.7 μm to 4.0 μm, particularly preferably 1.0 μm to 3.5 μm from the viewpoint of, for example, surface area and handleability.

The silica/alumina ratio in the zeolite with no transition metal supported or the transition metal-supported zeolite can be appropriately set, is not particularly limited, and is preferably 7 or more and 30 or less, more preferably 8 or more and 25 or less, further preferably 10 or more and 20 or less from the viewpoint of, for example, thermal durability and catalyst activity under a high temperature environment or after exposure to a high temperature. The aluminosilicate, in which the silica/alumina ratio is within the above preferable numerical value range, thus results in a tendency to obtain a catalyst or a catalyst carrier each having thermal durability and catalyst activity balanced at high levels.

On the other hand, the content of the transition metal in the transition metal-supported zeolite, which has a small pore size, is not particularly limited, and is preferably 0.1 to 10% by mass, more preferably 0.5 to 8% by mass based on the total amount.

The atomic ratio (transition metal/aluminum) of the transition metal to aluminum in the transition metal-supported zeolite, which has a small pore size, is not particularly limited, and is preferably 0.01 to 1.0, more preferably 0.05 to 0.7, further preferably 0.1 to 0.5.

As described above, the transition metal may be supported on the zeolite. Accordingly, one zeolite of the present embodiments is an AFX-type zeolite in which the SAR (SiO₂/Al₂O₃ ratio) is 10 or more and 30 or less, 2θ=21.77°±0.15° in an XRD chart obtained by powder X-ray diffraction analysis corresponds to a strongest line, the average particle size is 0.6 μm or more, and the transition metal is supported.

The transition metal-supported zeolite may be layered on a honeycomb carrier to provide a honeycomb stacked catalyst. The honeycomb stacked catalyst can be produced by, for example, wet coating a honeycomb carrier with an AFX-type zeolite on which a transition metal is supported, drying the resultant at 100 to 150° C., and calcining the resultant at 200 to 800° C. The amount of coating with the AFX-type zeolite is usually 10 to 1000 g, preferably 50 to 300 g, more preferably 80 to 200 g per liter of the honeycomb carrier.

One of the present embodiments relates to an AFX-type zeolite on which a transition metal is supported, and a honeycomb stacked catalyst including a honeycomb carrier, of the present embodiments.

### <Method for Producing Compound Represented by Formula (1) or Salt Thereof>

One production method of the present embodiments includes:
a step of providing a compound represented by formula (A) (step I);
a step of reacting the compound represented by the formula (A) with a hydrogen source by use of a catalyst, to thereby a compound represented by formula (2) (step II); and
a step of N-alkylating the compound represented by the formula (2) with an alkylation reagent (step III).

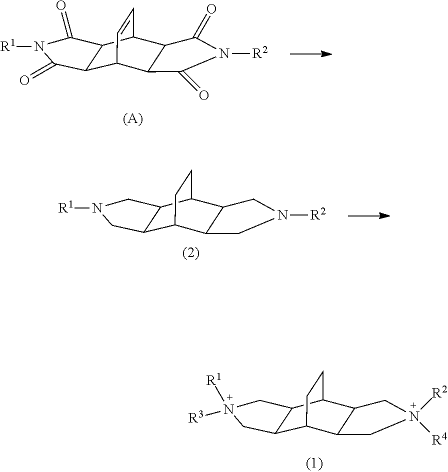

R¹ and R² in the formula (A) and the formula (2) are each independently an alkyl group. R¹ to R⁴ in the formula (1) are each independently an alkyl group.

### (Step I)

Step I is a step of providing a compound represented by formula (A). The compound represented by the formula (A) may be obtained as a commercially available product, or may be appropriately synthesized through a known synthesis route. For example, the compound may be obtained by synthesis with a reaction of commercially available bicyclo[2.2.2]oct-7-ene-2,3:5,6-tetracarboxylic dianhydride with an alkylamine or a salt thereof.

### (Step II)

Step II is a step of reacting the compound represented by the formula (A) with a hydrogen source by use of a catalyst, to thereby a compound represented by formula (2), and can be represented by the following scheme. The compound represented by the formula (A) is here also referred to as “N,N′-dialkylbicyclo[2.2.2]oct-7-ene-2,3:5,6-tetracarboxydiimide”.

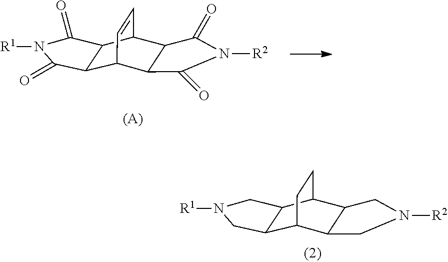

R¹ and R² in the scheme are each independently an alkyl group. R¹ and R² in the compounds represented by the formula (2) and the formula (A) have the same meanings as R¹ and R² in the formula (1), and preferable substituents can also include the same groups as in R¹ and R² in the formula (1).

The production method has no need for use of any strong reducing agent whose handling is difficult, for example, any reducing agent having the risk of ignition and the like, and therefore can safely and easily produce a compound represented by formula (2), N,N′-dialkylbicyclo[2.2.2]octane-2,3:5,6-dipyrrolidine. The production method then can allow for synthesis in relatively safe conditions and thus causes less facility burden and can allow for large-lot production, thereby allowing the resulting compound represented by the formula (2) to be enhanced in productivity and economic performance.

The hydrogen source used in the production method of the present embodiments can be appropriately selected from those capable of hydrogenating the compound represented by the formula (A). Specific examples include molecular hydrogen such as a hydrogen gas; and hydrogen donors such as ammonium formate, sodium formate, and hydrazine, but not particularly limited thereto. Among such hydrogen sources, molecular hydrogen is preferable.

The catalyst used in the production method of the present embodiments can be a catalyst usually usable in hydrogenation, and the type thereof is not particularly limited. The catalyst is preferably a heterogeneous catalyst. A heterogeneous catalyst is used to thereby facilitate an operation such as a post-treatment and allow the compound to be enhanced in productivity and economic performance even in large-lot production.

The catalyst is preferably a catalyst including a transition metal.

Examples of the transition metal include metals such as palladium (Pd), platinum (Pt), rhodium (Rh), vanadium (V), iron (Fe), cobalt (Co), nickel (Ni), copper (Cu), ruthenium (Ru), rhenium (Re), osmium (Os), molybdenum (Mo), and tungsten (W). Such metals may be used singly or in combinations of two or more kinds thereof.

The above-mentioned transition metal may be supported on a carrier. The carrier is not particularly limited as long as it is a carrier usually used as a catalyst carrier. Examples include inorganic oxide, activated carbon, and an ion-exchange resin. Examples of the inorganic oxide specifically include silica (SiO₂), titania (TiO₂), zirconia (Zro₂), alumina (Al₂O₃), magnesium oxide (MgO), tricalcium phosphate (HAP; hydroxyapatite), and any composite (for example, zeolite) of two or more of these inorganic oxides.

The amount of the catalyst used is not particularly limited, and is usually 0.01 to 10% by mol, preferably 0.1 to 5% by mol in terms of the amount of metal in the catalyst relative to the molar number of the compound represented by the formula (2).

The catalyst used in the production method can be suitably a catalyst where Pt and V are supported on a carrier. The catalyst where Pt and V are supported on a carrier can be used to thereby reduce the compound represented by the formula (2) in milder conditions.

The catalyst where Pt and V are supported on a carrier is also herein designated as “Pt—V/Z”. Here, Z represents the carrier.

Platinum constituting the Pt—V/Z is not particularly limited, and is preferably, for example, a platinum particle. The platinum particle is here a particle of at least one of metallic platinum or platinum oxide, preferably a particle of metallic platinum.

The platinum particle is not particularly limited as long as it contains at least platinum, and may include a small amount of noble metal(s) such as ruthenium, rhodium, and palladium.

The platinum particle may be a primary particle or a secondary particle. The average particle size of the platinum particle is preferably 1 to 30 nm, more preferably 1 to 10 nm. The average particle size refers to an average value of diameters of any number of particles observed with an electron microscope.

Vanadium constituting the Pt—V/Z is not particularly limited, and is preferably, for example, a vanadium oxide. Examples of the vanadium oxide include a vanadate ion (VO₄³⁻, VO₃³⁻), vanadium pentoxide, vanadium (II) oxide, and vanadium (IV) oxide. Among such vanadium oxides, V₂O₅ is preferable.

The compositional ratio of Pt and V in the Pt—V/Z is preferably 1:0.001 to 10, more preferably 1:0.005 to 5 in terms of respective number of moles, Pt as a metal: V as a metal.

The carrier Z in the Pt—V/Z is not particularly limited, and the adsorption ability thereof as a BET value may be 0.1 to 300 m²/g and the average particle size thereof may be 0.02 to 200 μm.

The shape of the carrier is not particularly limited, and examples thereof include a powder shape, a spherical particle shape, an amorphous granule shape, a cylindrical pellet shape, an extruded shape, and a ring shape.

The component constituting the carrier is preferably HAP as the above-mentioned carrier.

The Pt—V/Z can be produced by mixing a mixed liquid of a platinum compound and a vanadium compound with the carrier to obtain a mixture, and drying the mixture.

Examples of the platinum compound include platinum complex salts such as platinum acetylacetonate (Pt(acac)₂), tetraammine platinum (II) acetate, dinitrodiammine platinum (II), hexaammine platinum (II) carbonate, and bis(dibenzalacetone)platinum(0), and salts such as platinum chloride and potassium tetrachloroplatinate. Among such platinum compounds, Pt(acac)₂ is preferable.

Examples of the vanadium compound include vanadium complex salts such as vanadyl acetylacetonate (VO(acac)₂) and tetramethylammonium bis(tartrato)bis [oxovanadium (IV)] acid, and salts such as ammonium vanadate (V) and vanadium naphthenate. Among such vanadium compounds, VO(acac)₂ is preferable.

The mixed liquid in production of the Pt—V/Z is obtained by suspending or dissolving the platinum compound and the vanadium compound in a solvent. Examples of the solvent include water, and organic solvents such as alcohol and acetone. Such solvents can be used singly or in combinations of two or more kinds thereof.

The mixed liquid is mixed with the carrier. The method for mixing the mixed liquid with the carrier is not particularly limited as long as each of the components is sufficiently dispersed. The amount of the carrier in terms of metal, relative to 0.1 mmol of platinum, is preferably 0.1 to 100 g, more preferably 1 to 10 g. The carrier, after mixed, is preferably stirred for 0.5 to 12 hours.

The mixture of the mixed liquid with the carrier is dried after removal of the solvent by a rotary evaporator or the like. The drying is preferably, for example, drying at 80 to 200° C. for 1 to 60 hours. After the drying, a dried product is preferably, if necessary, pulverized and calcined by using a muffle furnace or the like.

The production method of the present embodiments can be specifically a method involving providing the compound represented by the formula (A) and mixing the compound with the catalyst and the hydrogen source to thereby allow for a reaction.

The mixing order of the compound represented by the formula (A), the catalyst, and the hydrogen source is any order. It is preferable in the production method of the present embodiments to mix the compound represented by the formula (A) and the catalyst, if necessary, add a solvent, and thereafter introduce the hydrogen source into a reactor, from the viewpoint of workability.

The reaction in the production method of the present embodiments is allowed to progress in low-temperature and low-pressure conditions, and thus molecular sieves may be added into the reaction system. The amount of the molecular sieves added is preferably 0.1 to 10 times, more preferably 0.5 to 5 times the mass of the compound represented by the formula (A).

Step II in the present embodiments may be performed in the presence of a solvent, namely, under a wet process.

The solvent is not particularly limited as long as the solvent can dissolve the compound represented by the formula (A), and may be appropriately selected depending on, for example, the reaction temperature and the reactants.

Examples of the solvent include water; aromatic hydrocarbon-based solvents such as benzene and toluene; amide-based solvents such as acetonitrile, N,N-dimethylacetamide, and N,N-dimethylformamide; ether-based solvents such as tetrahydrofuran (hereinafter, also designated as “THE”), diethyl ether, and 1,2-dimethoxyethane; alcohol-based solvents such as methanol, ethanol, and isopropanol; and halogen-based solvents such as dichloromethane, dichloroethane, and chloroform. Such solvents can be used singly or in any combination of two or more kinds thereof at any ratio.

Among such solvents, an ether-based solvent is preferable, and 1,2-dimethoxyethane is more preferable.

Use of the solvent and the amount thereof used may be appropriately set in consideration of other reaction conditions, and are not particularly limited, and the concentration of the compound represented by the formula (A) in the reaction mixture is preferably 0.001 to 10 mol/L, more preferably 0.01 to 5 mol/L, further preferably 0.01 to 3 mol/L.

The amount of the catalyst used is preferably 0.1 to 50 times, preferably 0.5 to 20 times, further preferably 1 to 10 times the mass of the compound represented by the formula (A).

The reaction temperature is not particularly limited, and is usually in the range from 20 to 200° C., preferably in the range from 50 to 150° C., more preferably in the range from 50 to 120° C.

The reaction time may be appropriately adjusted by monitoring progress of the reaction by use of, for example, GC-MS, and is usually 1 minute to 100 hours, preferably 0.5 hours to 70 hours, more preferably 1 hour to 60 hours.

In a case where molecular hydrogen is used in the production method of the present embodiments, the hydrogen pressure in the reactor is usually 0.1 to 10 MPa, preferably 1.0 to 10 MPa, more preferably 2.0 to 8.0 MPa.

In a case where the solvent is used in the reaction, the mixture after completion of the reaction in step II, which is in the form of a reaction solution obtained, may be, if necessary, concentrated and thereafter the residue may be used as a raw material, a precursor, or an intermediate, as it is, or the reaction mixture may be appropriately subjected to a post-treatment to obtain the compound represented by the formula (2). Specific methods for the post-treatment can include known purification methods such as water washing, filtration, drying, extraction, distillation, and chromatography. Such purification methods may be performed in combinations of two or more kinds thereof.

### (Step III)

Step III is a step of N-alkylating the compound represented by the formula (2) with an alkylation reagent, and can be represented by the following scheme. The following compound represented by formula (2) is herein also referred to as “N,N′-dialkylbicyclo[2.2.2]octane-2,3:5,6-dipyrrolidine”.

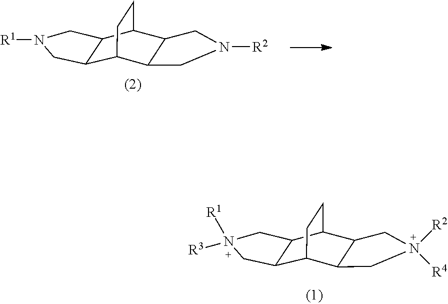

N,N,N′,N′-tetraethylbicyclo[2.2.2]oct-7-ene-2,3:5,6-dipyrrolidinium hydroxide has been used in synthesis of a zeolite in a conventional art (see U.S. Patent Application Publication No. 6049018 (Patent Literature 1) and Japanese Patent Laid-Open No. 2016-169139 (Patent Literature 2)). In synthesis of the hydroxide, a solution of the hydroxide is obtained by exchanging an ion of N,N,N′,N′-tetraethylbicyclo[2.2.2]oct-7-ene-2,3:5,6-dipyrrolidiniumiodide obtained as a solid powder with a hydroxide-type negative ion-exchange resin, and concentrating the resultant.

On the other hand, the compound (salt) of the present embodiments, which is in the form of a salt obtained by alkylation as described above, namely, a salt where X is a counter anion, can be used as it is, for synthesis of a zeolite. Accordingly, in this case, the trouble for preparation of the hydroxide can be avoided, and a zeolite can be efficiently produced. For example, in the case of use as the hydroxide, for example, ion-exchange with a hydroxide-type negative ion-exchange resin and concentration may be performed similarly to those conventionally performed.

### <Method for Producing AFX-Type Zeolite>

One of the present embodiments relates to a method for producing an AFX-type zeolite, and the production method includes at least a step of preparing a mixture including at least a silica and alumina source, an organic structure directing agent (OSDA) including a compound represented by the following (1) and/or a salt thereof, an alkali metal hydroxide, and water, and a step of hydrothermally treating the mixture to synthesize an AFX-type zeolite:

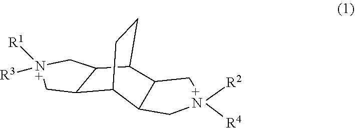

wherein R¹ to R⁴ are each independently an alkyl group.

The AFX-type zeolite of the present embodiments can be obtained by the method for producing an AFX-type zeolite of the present embodiments.

### <AFX-Type Zeolite Having Macropore>

One of the present embodiments relates to an AFX-type zeolite having a macropore. Such a macropore in the present embodiments is according to the definition of IUPAC. Specifically, such a macropore refers to a pore having a diameter of more than 50 nm. The presence of such a macropore in the AFX-type zeolite can be determined from an SEM image of the zeolite.

An AFX-type zeolite having such a macropore, of the present embodiments, can be produced by, for example, using an organic structure directing agent (OSDA) including a compound represented by formula (1) and/or a salt thereof. The AFX-type zeolite having such a macropore, of the present embodiments, can be specifically produced by the above-mentioned method for producing an AFX-type zeolite. In other words, the AFX-type zeolite can be produced by a production method including at least a step of preparing a mixture including at least a silica and alumina source, an organic structure directing agent (OSDA) including a compound represented by the following (1) and/or a salt thereof, an alkali metal hydroxide, and water, and a step of hydrothermally treating the mixture to synthesize an AFX-type zeolite.

### EXAMPLES

Hereinafter, features of the present invention are more specifically described with reference to Examples and Comparative Examples, but the present invention is not limited thereto at all. In other words, materials, amounts used, percentages, treatment contents, treatment procedures, and the like shown in Examples below can be appropriately modified without departing from the gist of the present invention. The values with respect to various production conditions and evaluation results in Examples below each have the meaning of a preferable upper limit value or a preferable lower limit value in an aspect for carrying out the present invention, and each preferable range thereof may be any range defined by a combination of the value of the upper limit or lower limit and any value in Examples described below, or a combination of values of Examples.

Any Example and any Comparative Example according to a specific aspect in a first group are respectively designated as “Example A” and “Comparative Example A”. Any Production Example, any Example and any Reference Example according to a specific aspect in a second group are respectively designated as “Production Example B”, “Example B” and “Reference Example B”. Any Production Example, any Example and any Comparative Example according to a specific aspect in a third group are respectively designated as “Production Example C”, “Example C” and “Comparative Example C”. Any Example according to a specific aspect in a fourth group is encompassed in Examples according to specific aspects in first to third groups.

### Example A1: synthesis of N,N,N′,N′-tetraethylbicyclo[2.2.2]octane-2,3:5,6-dipyrrolidinium diiodide

In 1,200 mL of an isopropyl alcohol (IPA)-modified alcohol was dissolved 370.0 g of N,N′-diethylbicyclo[2.2.2]oct-7-ene-2,3:5,6-dipyrrolidine (molecular weight 246.39) synthesized according to Patent Literature 2, and 31.08 g (corresponding to 1.0% by mol of a substrate as palladium) of a 5% palladium carbon catalyst (water-containing K-type product manufactured by N.E. Chemcat Corporation), in terms of dry mass, was added thereto to allow a reaction to occur by hydrogen at 50° C. and an ordinary pressure for 190 hours. The percentage of conversion of the substrate according to gas chromatography (GC) was 99% or more. After the catalyst was removed by separation with filtration, 516.0 g (molecular weight 155.11, 2.2 equivalents) of ethyl iodide was dropped with stirring. The resultant was mildly refluxed in a nitrogen atmosphere for 16 hours, thereafter cooled and then filtered, and washed with acetone and dried, to thereby obtain 703.0 g (yield 90%) of a white powder of an objective substance, N,N,N′,N′-tetraethylbicyclo[2.2.2]octane-2,3:5,6-dipyrrolidinium diiodide.

¹H-NMR and ¹³C-NMR of the white powder obtained are shown below.

¹H-NMR (400 MHZ, D₂O) δ: 3.82 (dd, 4H), 3.49 (q4, 4H), 3.38 (q4, 4H), 3.33 (d, 4H), 2.69 (m, 4H), 1.80 (s, 2H), 1.64 (s, 4H), 1.36 (t, 6H), 1.31 (t, 6H). ¹³C-NMR (100 Hz, D₂O) δ: 65.00 (×4), 58.51 (×2), 54.41 (×2), 40.11 (×4), 28.33 (×2), 14.86 (×2), 11.01 (×2), 10.17 (×2)

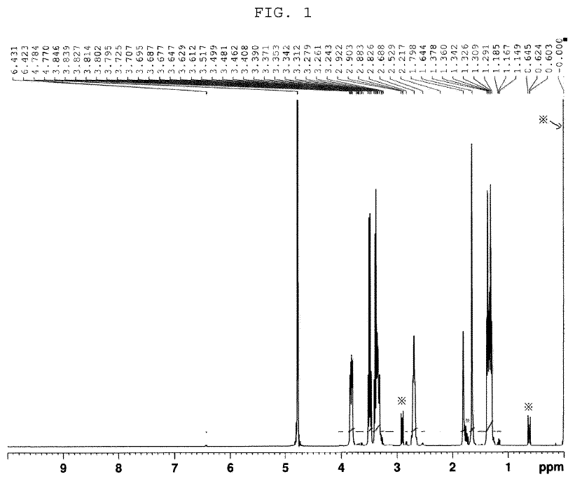

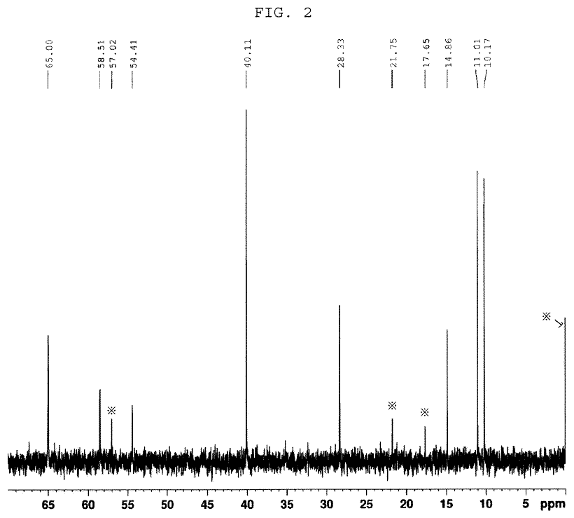

¹H-NMR spectral data and ¹³C-NMR spectral data of a D₂O solution of N,N,N′,N′-tetraethylbicyclo[2.2.2]octane-2,3:5,6-dipyrrolidinium diiodide are respectively illustrated in FIG. 1 and FIG. 2.

Conditions of the gas chromatography were here as follows.

Apparatus name: GCMS-QP2010 (manufactured by Shimadzu Corporation)
Column: SH-Rtx-200 MS manufactured by Shimadzu Corporation
Carrier gas: helium
Total flow rate: 98.9 mL/min
Flow rate in column: 2.56 mL/min
Temperature: the temperature of a column oven was raised from 40° C. to 300° C. at 10° C./min and thereafter retained at 300° C. for 10 minutes.

Measurement conditions of the NMR were as follows.

Apparatus name: Ascend 4000 (manufactured by Bruker Japan K.K.)

Measurement method: 1H-NMR and 13C-NMR were measured by dissolving a sample in deuterated water.

### Example A2: synthesis of AFX-type zeolite

In a polyethylene beaker were stirred 28.0 g of N,N,N′,N′-tetraethylbicyclo[2.2.2]octane-2,3:5,6-dipyrrolidinium diiodide (molecular weight 558.62), 116.0 g of a 4.8% by mass sodium hydroxide solution, 37.5 g of FAU-type zeolite CBV712 (manufactured by Zeolyst C.V., silica/alumina ratio SAR: 10.9), and 47.0 g of water for 48 hours. The compositional ratio in the mixture was as follows.

TABLE 1
SiO₂

| | |
|---|---|
| 0.092 | Al₂O₃ |
| 0.107 | OSDA |
| 0.152 | Na₂O |
| 19.94 | H₂O |

The numerical value of each of the components in the mixture means the molar number ratio with the molar number of SiO₂ as 1.

Next, the starting material composition (mixture) was placed in a 300-cc stainless sealed pressure-resistant container with an inner cylinder of Teflon (registered trademark), and left to stand still and retained at 170° C. for 40 hours. A product after this hydrothermal treatment was subjected to solid-liquid separation, and the resulting solid phase was washed with a sufficient amount of water and dried at 105° C., to thereby obtain a product. Powder X-ray diffraction analysis was performed and it was thus confirmed that the product was a single phase of AFX-type zeolite.

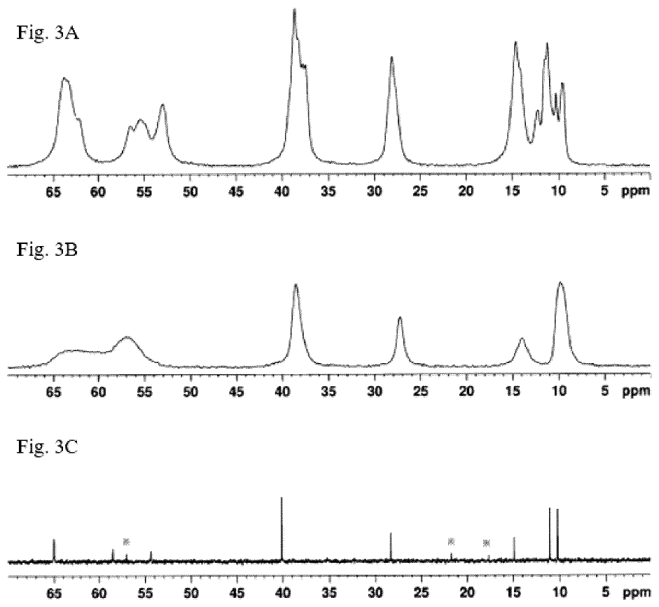

FIG. 3B illustrates solid NMR spectral data of an AFX-type zeolite obtained using N,N,N′,N′-tetraethylbicyclo[2.2.2]octane-2,3:5,6-dipyrrolidinium. FIG. 3A illustrates solid NMR spectral data of N,N,N′,N′-tetraethylbicyclo[2.2.2]octane-2,3:5,6-dipyrrolidinium, and FIG. 3C illustrates ¹³CNMR spectral data of a D₂O solution. In FIG. 3C, * indicates a peak of an internal standard (4,4-dimethyl-4-silapentane-1-sulfonic acid).

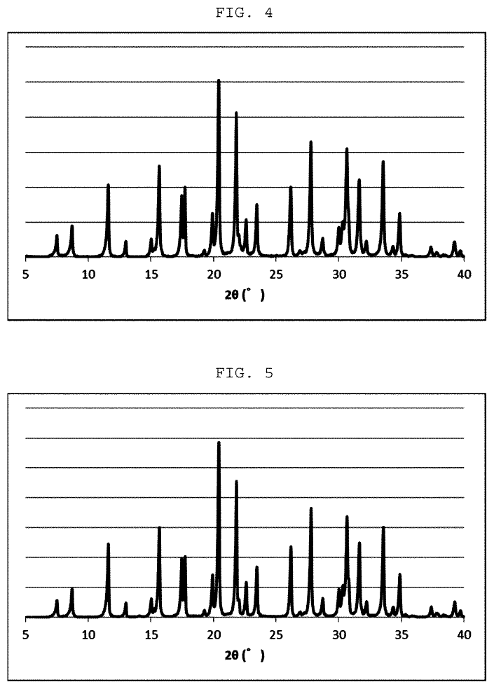

FIG. 4 illustrates an XRD chart of the AFX-type zeolite obtained by Example A2.

Measurement conditions of powder X-ray diffraction were here as follows.

Apparatus name: X'Pert Pro (manufactured by Spectris)

Measurement method: a powdery measurement sample was packed in a grooved glass sample plate container and subjected to measurement. The measurement was performed at a tube voltage of 45 kV and a tube current of 40 mA with a CuKα ray as an X-ray source.

### Example A3: synthesis of AFX-type zeolite

In 800 mL of water was dissolved 120.0 g of N,N,N′,N′-tetraethylbicyclo[2.2.2]octane-2,3:5,6-dipyrrolidinium diiodide (molecular weight 558.62), 800.0 g of Diaion SA10AOH (manufactured by Mitsubishi Chemical Corporation) was loaded thereto, and the resultant was stirred at room temperature for 48 hours. After filtration and washing, concentration was made until the total mass of a filtrate and a washing liquid was 379.9 g, to thereby obtain 19.26% by mass of N,N,N′,N′-tetraethylbicyclo[2.2.2]octane-2,3:5,6-dipyrrolidinium dihydroxide (molecular weight 340.55).

To 6.5 g of the solution were added 6.2 g of a 4.8% sodium hydroxide solution, 4.0 g of FAU-type zeolite CBV712 (manufactured by Zeolyst C.V., silica/alumina ratio SAR: 10.9), and 5.8 g of water, and the resultant was stirred in a polyethylene beaker for 72 hours. The compositional ratio in the mixture was as follows.

TABLE 2
SiO₂

| | |
|---|---|
| 0.092 | Al₂O₃ |
| 0.077 | OSDA |
| 0.078 | Na₂O |
| 20.74 | H₂O |

The numerical value of each of the components in the mixture means the molar number ratio with the molar number of SiO₂ as 1.

Next, the starting material composition (mixture) was placed in a 50-cc stainless sealed pressure-resistant container with an inner cylinder of Teflon, and left to stand still and retained at 170° C. for 96 hours. A product after this hydrothermal treatment was subjected to solid-liquid separation, and the resulting solid phase was washed with a sufficient amount of water and dried at 105° C., to thereby obtain a product. Powder X-ray diffraction analysis was performed and it was thus confirmed that the product was a single phase of AFX-type zeolite.

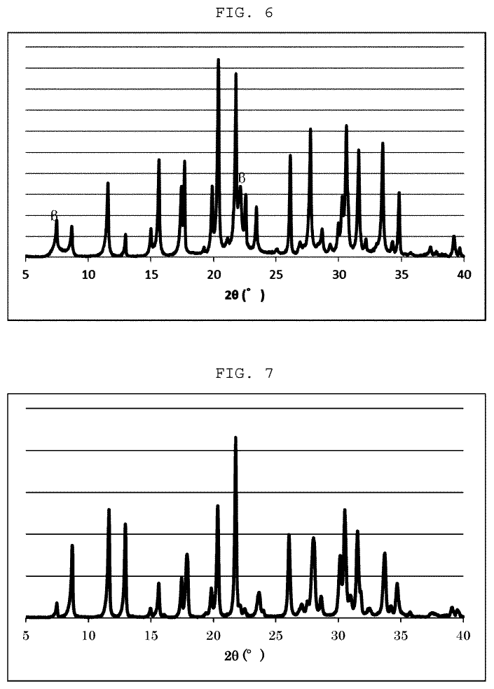

FIG. 5 illustrates an XRD chart of the AFX-type zeolite obtained by Example A3.

Table 3 shows each main peak position, and Table 4 shows the relative intensity of each peak in XRD of each of the AFX-type zeolites obtained in Examples A2 and A3, with, as 1, the integral intensity of the strongest peak (2θ=) 20.42° of the AFX-type zeolite obtained in Example A2.

TABLE 3

| Peak position | Example A2 | Example A3 |
|---|---|---|
| 2θ (deg) | 2θ | 2θ |
| 7.50 ± 0.15 | 7.49 | 7.50 |
| 8.71 ± 0.15 | 8.72 | 8.72 |
| 11.60 ± 0.15 | 11.60 | 11.61 |
| 13.01 ± 0.15 | 13.01 | 13.02 |
| 15.67 ± 0.15 | 15.68 | 15.69 |
| 17.46 ± 0.15 | 17.46 | 17.47 |
| 17.72 ± 0.15 | 17.73 | 17.74 |
| 19.93 ± 0.15 | 19.91 | 19.94 |
| 20.42 ± 0.15 | 20.42 | 20.43 |
| 21.84 ± 0.15 | 21.83 | 21.85 |
| 23.47 ± 0.15 | 23.47 | 23.49 |
| 26.19 ± 0.15 | 26.19 | 26.20 |
| 27.79 ± 0.15 | 27.79 | 27.82 |
| 30.67 ± 0.15 | 30.66 | 30.68 |
| 31.65 ± 0.15 | 31.65 | 31.66 |
| 33.56 ± 0.15 | 33.55 | 33.59 |

TABLE 4

| Peak position | Example A2 | Example A3 |
|---|---|---|
| 2θ (deg) | 2θ | 2θ |
| 7.50 ± 0.15 | 0.157 | 0.147 |
| 8.71 ± 0.15 | 0.230 | 0.219 |
| 11.60 ± 0.15 | 0.437 | 0.467 |
| 13.01 ± 0.15 | 0.093 | 0.086 |
| 15.67 ± 0.15 | 0.456 | 0.507 |
| 17.46 ± 0.15 | 0.340 | 0.338 |
| 17.72 ± 0.15 | 0.312 | 0.312 |
| 19.93 ± 0.15 | 0.160 | 0.224 |
| 20.42 ± 0.15 | 1.000 | 0.853 |
| 21.84 ± 0.15 | 0.806 | 0.801 |
| 23.47 ± 0.15 | 0.306 | 0.317 |
| 26.19 ± 0.15 | 0.410 | 0.425 |
| 27.79 ± 0.15 | 0.511 | 0.523 |
| 30.67 ± 0.15 | 0.583 | 0.422 |
| 31.65 ± 0.15 | 0.444 | 0.376 |
| 33.56 ± 0.15 | 0.612 | 0.484 |

The compositional ratio except water in the AFX-type zeolite obtained in Example A2 was the following compositional ratio.
M_{a/b}Q_{c}Si_{48-d}Al_{d}O₉₆

In the formula, M represents a metal cation, a represents 1 to 10, b represents a valence of M, Q represents N,N,N′,N′-tetraethylbicyclo[2.2.2]octane-2,3:5,6-dipyrrolidinium cation, c represents 0.5 to 2, and d represents 4 to 12.

The compositional ratio except water in the AFX-type zeolite obtained in Example A2 specifically satisfied a=5.0, b=1.0, c=1.3, and d=7.6.

### Comparative Example A1: synthesis of AFX-type zeolite

A product was obtained in the same manner as in Example A1 except that 2.0 g of N,N,N′,N′-tetraethylbicyclo[2.2.2]oct-7-ene-2,3:5,6-dipyrrolidinium diiodide (molecular weight 556.61) synthesized according to the method of Patent Literature 2 was used instead of 2.0 g of N,N,N′,N′-tetraethylbicyclo[2.2.2]octane-2,3:5,6-dipyrrolidinium diiodide (molecular weight 558.62). Powder X-ray diffraction analysis was performed and it was thus confirmed that not only an AFX-type zeolite, but also a beta-type zeolite was produced in the product.

FIG. 6 illustrates an XRD chart of the AFX-type zeolite obtained by Comparative Example A1.

### Example A4: AFX-Type Zeolite Production Involving Calcination Step

The amount of N,N,N′,N′-tetraethylbicyclo[2.2.2]octane-2,3:5,6-dipyrrolidinium diiodide used was 3.0 g, the amount of a 4.8% by mass sodium hydroxide solution used was 12.7 g, the amount of FAU-type zeolite CBV712 used was 4.2 g and the amount of water used was 2.7 g, and these were stirred in a polyethylene beaker for 48 hours. The compositional ratio in the mixture was as follows.

TABLE 5
SiO₂

| | |
|---|---|
| 0.092 | Al₂O₃ |
| 0.102 | OSDA |
| 0.148 | Na₂O |
| 16.96 | H₂O |

The numerical value of each of the components in the mixture means the molar number ratio with the molar number of SiO₂ as 1.

Next, the starting material composition (mixture) was placed in a 50-cc stainless sealed pressure-resistant container with an inner cylinder of Teflon, and left to stand still and retained at 170° C. for 40 hours. A product after this hydrothermal treatment was subjected to solid-liquid separation, and the resulting solid phase was washed with a sufficient amount of water and dried at 105° C., to thereby obtain a product. Next, the temperature was raised to 600° C. at a rate of temperature rise of 1° C./min, and thereafter calcination was made for 5 hours. Powder X-ray diffraction analysis of the product obtained was performed and it was thus confirmed that the product was a single phase of AFX-type zeolite.

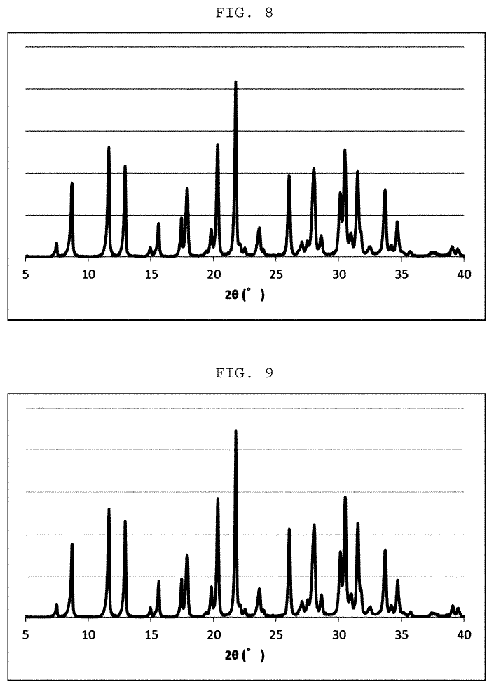

FIG. 7 illustrates an XRD chart of the AFX-type zeolite obtained by Example A4.

### Example A5: AFX-Type Zeolite Production Involving Calcination Step

The amount of N,N,N′,N′-tetraethylbicyclo[2.2.2]octane-2,3:5,6-dipyrrolidinium diiodide used was 310.0 g, the amount of a 4.8% by mass sodium hydroxide solution used was 1310.0 g, the amount of FAU-type zeolite CBV712 used was 425.0 g and the amount of water used was 335.0 g, and these were stirred in a polyethylene beaker for 48 hours. The compositional ratio in the mixture was as follows.

TABLE 6
SiO₂

| | |
|---|---|
| 0.092 | Al₂O₃ |
| 0.105 | OSDA |
| 0.151 | Na₂O |
| 17.78 | H₂O |

The numerical value of each of the components in the mixture means the molar number ratio with the molar number of SiO₂ as 1.

Next, 1450 g of the starting material composition (mixture) was placed in a 1-L stainless autoclave, and stirred at 300 rpm and retained at 170° C. for 60 hours. A product after this hydrothermal treatment was subjected to solid-liquid separation, and the resulting solid phase was washed with a sufficient amount of water and dried at 105° C., to thereby obtain a product. Next, the temperature was raised to 600° C. at a rate of temperature rise of 1° C./min, and thereafter calcination was made for 5 hours. The SAR (SiO₂/Al₂O₃ ratio) in terms of solid content of the powder obtained, as measured by fluorescent X-ray analysis, was 10.7.

Measurement conditions of powder X-ray diffraction were here as follows.

The apparatus used in the fluorescent X-ray analysis was Axios (Spectris, Panalytical department). Into a vinyl chloride ring was placed 5 g of a measurement sample, and the sample was molded under pressure at a load of 20 t and then subjected to measurement. The analysis software used here was UniQuant5. Powder X-ray diffraction analysis was performed and it was thus confirmed that the product was a single phase of AFX-type zeolite.

FIG. 8 illustrates an XRD chart of the AFX-type zeolite obtained by Example A5.

### Example A6: AFX-Type Zeolite Production Involving Calcination Step

To 9.0 g of a solution of 19.26% by mass of N,N,N′,N′-tetraethylbicyclo[2.2.2]octane-2,3:5,6-dipyrrolidinium dihydroxide (molecular weight 340.55) obtained in Example A3 were added 2.5 g of a 4.8% sodium hydroxide solution, 4.1 g of FAU-type zeolite CBV712 (manufactured by Zeolyst C.V., silica/alumina ratio SAR: 10.9), 1.0 g of sodium chloride, and 5.8 g of water, and the resultant was stirred in a polyethylene beaker for 48 hours. The compositional ratio in the mixture was as follows.

TABLE 7
SiO₂

| | |
|---|---|
| 0.092 | Al₂O₃ |
| 0.104 | OSDA |
| 0.206 | Na₂O |
| 20.01 | II₂O |
| 0.350 | HCl |

The numerical value of each of the components in the mixture means the molar number ratio with the molar number of SiO₂ as 1.

Next, the starting material composition (mixture) was placed in a 50-cc stainless sealed pressure-resistant container with an inner cylinder of Teflon, and left to stand still and retained at 155° C. for 240 hours. A product after this hydrothermal treatment was subjected to solid-liquid separation, and the resulting solid phase was washed with a sufficient amount of water and dried at 105° C., to thereby obtain a product. Next, the temperature was raised to 600° C. at a rate of temperature rise of 1° C./min, and thereafter calcination was made for 5 hours. Powder X-ray diffraction analysis was performed and it was thus confirmed that the product was a single phase of AFX-type zeolite.

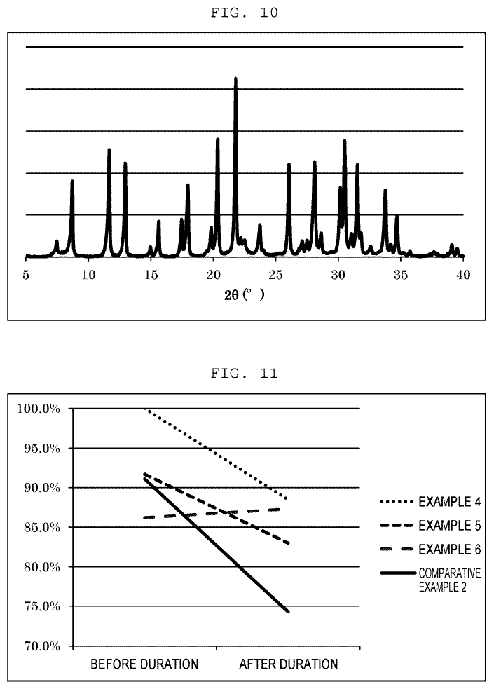

FIG. 9 illustrates an XRD chart of the AFX-type zeolite obtained by Example A6.

### Comparative Example A2: AFX-Type Zeolite Production Involving Calcination Step

In a polyethylene beaker were stirred 7.7 g of 17.4% by mass N,N,N′,N′-tetraethylbicyclo[2.2.2]oct-7-ene-2,3:5,6-dipyrrolidinium dihydroxide (molecular weight 338.53) synthesized according to the method of Patent Literature 2, instead of the solution of N,N,N′,N′-tetraethylbicyclo[2.2.2]octane-2,3:5,6-dipyrrolidinium dihydroxide, 3.1 g of a 4.8% sodium hydroxide solution, 3.0 g of FAU-type zeolite CBV712 (manufactured by Zeolyst C.V., silica/alumina ratio SAR: 10.9), and 3.5 g of water for 48 hours. The compositional ratio in the mixture was as follows.

TABLE 8
SiO₂

| | |
|---|---|
| 0.092 | Al₂O₃ |
| 0.108 | OSDA |
| 0.051 | Na₂O |
| 20.20 | H₂O |

The numerical value of each of the components in the mixture means the molar number ratio with the molar number of SiO₂ as 1.

Next, the starting material composition (mixture) was treated in the same manner as in Example A6, to thereby obtain a product. Powder X-ray diffraction analysis was performed and it was thus confirmed that the product was a single phase of AFX-type zeolite.

FIG. 10 illustrates an XRD chart of the AFX-type zeolite obtained by Comparative Example A2.

### <Measurement of Hydrothermal Durability>

The hydrothermal durability was measured by weighing 2.0 g of each of the AFX-type zeolite powders of Examples A4, A5 and A6, and Comparative Example A2, in a crucible, placing the resultant in an electric furnace (trade name OXK-600X, manufactured by Kyoei Electric Kilns Co., Ltd.) to which a gas humidification apparatus (trade name RMG-1000, manufactured by J-Science Lab Co., Ltd.) was connected, and retaining the resultant at 750° C. for 40 hours under supply of air including 10% steam at a flow rate of 70 L/min.

Table 9 shows the change in integral intensity of each peak in XRD of each sample before and after measurement of the hydrothermal durability. The numerical value of each peak intensity is a relative value with the integral intensity of the strongest peak (2θ=) 21.77° of the AFX-type zeolite obtained in Example A4, before measurement of the hydrothermal durability, as 1.0. The lowest column in the Table shows a relative value of the total integral intensity in each of Examples and Comparative Examples with the total integral intensity in Example A4 before measurement of the hydrothermal durability, as 100%.

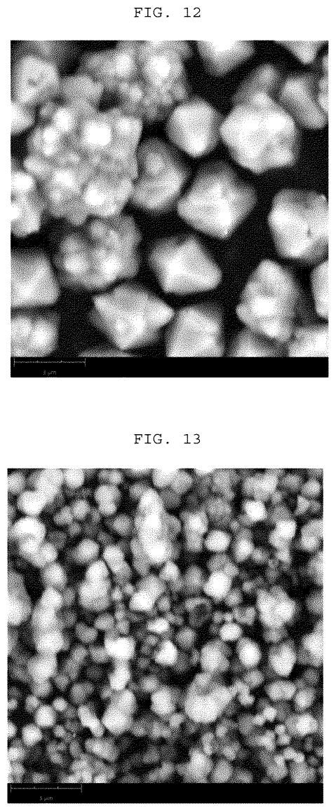

FIG. 11 illustrates the change in total integral intensity of XRD peaks before and after measurement of the hydrothermal durability of each sample. As illustrated in FIG. 11, the slope of the change in numerical value in each of Examples A4, A5 and A6, before and after the measurement, was suppressed as compared with that in Comparative Example A2, and it was thus found that the hydrothermal durability was excellent.

TABLE 9

| | | | | | | | | Comparative | |
|---|---|---|---|---|---|---|---|---|---|
| | | Example A4 | | Example A5 | | Example A6 | | Example A2 | |
| Peak position | | Before | After | Before | After | Before | After | Before | After |
| 2 θ | (deg) | duration | duration | duration | duration | duration | duration | duration | duration |
| 7.46 | ±0.15 | 0.1102 | 0.0723 | 0.1160 | 0.1250 | 0.0856 | 0.1514 | 0.1354 | 0.1156 |
| 8.69 | ±0.15 | 0.4859 | 0.5790 | 0.5078 | 0.5857 | 0.4699 | 0.5979 | 0.4990 | 0.4911 |
| 11.64 | ±0.15 | 0.7171 | 0.7506 | 0.6891 | 0.7450 | 0.6616 | 0.7626 | 0.6794 | 0.6677 |
| 12.93 | ±0.15 | 0.5679 | 0.5643 | 0.5447 | 0.5549 | 0.5544 | 0.5792 | 0.5208 | 0.4674 |
| 15.60 | ±0.15 | 0.2239 | 0.2187 | 0.2401 | 0.2221 | 0.2241 | 0.2395 | 0.2221 | 0.2163 |
| 17.43 | ±0.15 | 0.1383 | 0.2498 | 0.2178 | 0.3442 | 0.1856 | 0.2158 | 0.1928 | 0.1219 |
| 17.90 | ±0.15 | 0.4183 | 0.4059 | 0.4864 | 0.3429 | 0.4262 | 0.5407 | 0.4909 | 0.4602 |
| 19.81 | ±0.15 | 0.1539 | 0.1437 | 0.1340 | 0.1232 | 0.1135 | 0.1613 | 0.2106 | 0.0973 |
| 20.32 | ±0.15 | 0.7353 | 0.7180 | 0.5992 | 0.6040 | 0.5781 | 0.7671 | 0.6826 | 0.5208 |
| 21.77 | ±0.15 | 1.0000 | 0.9554 | 0.9712 | 0.7668 | 0.9624 | 1.0074 | 1.0025 | 0.5668 |
| 23.67 | ±0.15 | 0.2505 | 0.1719 | 0.2253 | 0.2095 | 0.2548 | 0.3262 | 0.2390 | 0.1586 |
| 26.03 | ±0.15 | 0.5317 | 0.4927 | 0.5366 | 0.4190 | 0.4515 | 0.3476 | 0.5510 | 0.4114 |
| 28.05 | ±0.15 | 0.9259 | 0.6860 | 0.7774 | 0.5677 | 0.9182 | 0.5621 | 0.5839 | 0.5801 |
| 30.49 | ±0.15 | 0.8991 | 0.6191 | 0.7605 | 0.6177 | 0.6202 | 0.5312 | 0.7008 | 0.5240 |
| 31.50 | ±0.15 | 0.6756 | 0.3638 | 0.4145 | 0.3598 | 0.3453 | 0.2913 | 0.4510 | 0.4735 |
| 33.71 | ±0.15 | 0.5745 | 0.4476 | 0.4884 | 0.3906 | 0.3999 | 0.2613 | 0.5012 | 0.3758 |

| | | | | | | | | |
|---|---|---|---|---|---|---|---|---|
| Total integral intensity | 8.407975 | 7.438838 | 7.709169 | 6.978148 | 7.251408 | 7.342419 | 7.663212 | 6.248479 |
| Relative value of total | 100.0% | 88.5% | 91.7% | 83.0% | 86.2% | 87.3% | 91.1% | 74.3% |
| integral intensity | | | | | | | | |

### <Sem Image>

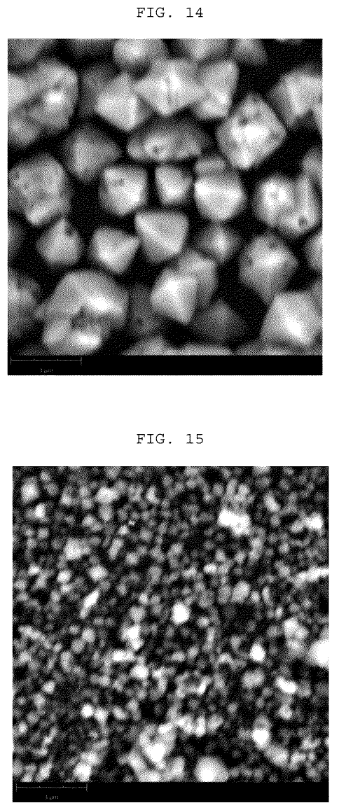

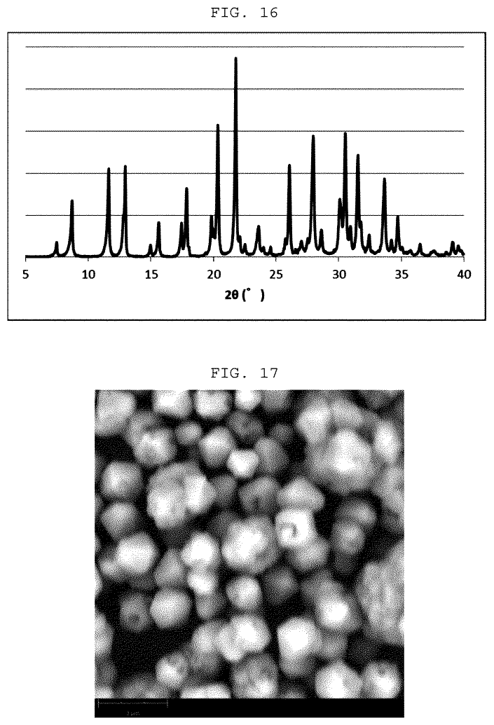

SEM images of the AFX-type zeolites obtained by Example A4, Example A5, Example A6, and Comparative Example A2 are respectively illustrated in FIG. 12, FIG. 13, FIG. 14, and FIG. 15.

The AFX-type zeolites of Example A4, Example A5, and Example A6 respectively had average particle sizes of about 3.84 μm, about 0.70 μm, and about 3.13 μm, and respectively had coefficients of variation of 30.4%, 36.9%, and 24.9%. The median particle sizes were respectively 3.60 μm, 0.67 μm, and 3.15 μm. In this regard, the AFX-type zeolite of Comparative Example A2 had an average particle size of 0.3 μm, a coefficient of variation of 55.6%, and a median particle size of 0.45 μm.

The SAR was 10 to 30, 2θ=21.77°±0.15° corresponded to a strongest line and the average particle size was 0.6 μm or more, and therefore the hydrothermal durability was considered to be enhanced.

The average particle size was here obtained by taking an image at a magnification of 6000× in a condition of an acceleration voltage of 10 kV with a scanning electron microscope (SEM, manufactured by Phenom-World B.V.), selecting any 100 particles in the image taken, and measuring the longest size of each of such particles. The average particle size was defined as the average value with respect to the longest size of each of such particles, and the median particle size was defined as the median value.

Each of the AFX-type zeolites of Example A4, Example A5, and Example A6 included a particle having a macropore. Such each AFX-type zeolite, which included a particle having a macropore, was confirmed from each of the SEM images.

### Example A7: AFX-Type Zeolite Production Involving Calcination Step and Ion-Exchange Step

In each of four 300-ml stainless sealed pressure-resistant containers, with an inner cylinder of Teflon, was placed 930 g of the starting material composition (mixture) obtained in Example A5, and the resultant was retained at 170° C. for 40 hours. A product after this hydrothermal treatment was subjected to solid-liquid separation, and the resulting solid phase was washed with a sufficient amount of water and dried at 105° C., to thereby obtain a product. Next, the temperature was raised to 600° C. at a rate of temperature rise of 1° C./min, and thereafter calcination was made for 5 hours. After ion-exchange using an aqueous ammonium nitrate solution including ammonium nitrate in the same amount as above and water in an amount of 10 times the same amount was repeated three times, the resultant was washed with a sufficient amount of pure water and dried at 120° C., to thereby obtain a NH₄⁺-type AFX-type zeolite.

### (Supporting of Cu)

After 120.0 g of the NH₄⁺-type AFX-type zeolite obtained was impregnated with a mixture of 36.0 g of an aqueous 50% copper nitrate trihydrate solution and 30.0 g of water, the resultant was dried at 100 to 120° C. The resultant was impregnated with a mixture of 7.0 g of morpholine and 35 g of water under an environment of 25° C., and again dried at 100 to 120° C., to thereby obtain a Cu-supported AFX-type zeolite of Example A7. The amount of supporting of Cu in terms of solid content, as measured by fluorescent X-ray analysis, was 4.22% by mass, and the SAR (SiO₂/Al₂O₃ ratio) was 10.7. Powder X-ray diffraction analysis of the product obtained was performed and it was thus confirmed that the product was a single phase of AFX-type zeolite.

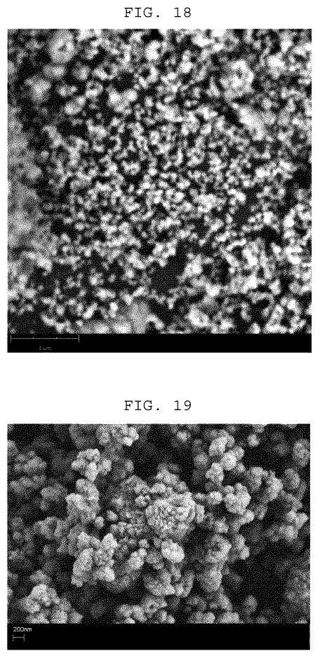

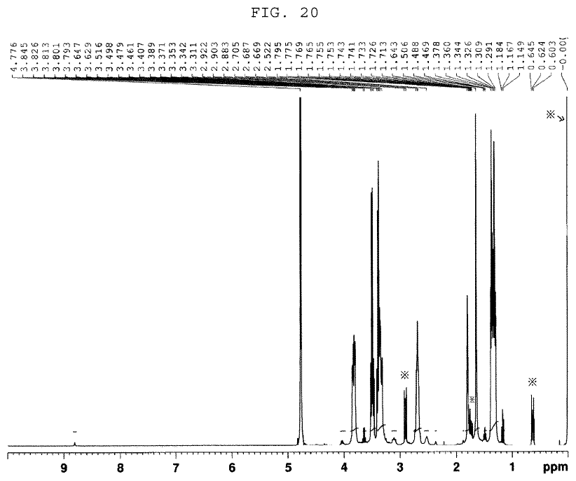

FIG. 16 illustrates an XRD chart of the Cu-supported AFX-type zeolite of Example A7, and FIG. 17 illustrates an SEM image of the Cu-supported AFX-type zeolite of Example A7. The Cu-supported AFX-type zeolite of Example A7 had an average particle size of about 2 μm. The AFX-type zeolite of Example A7 included a particle having a macropore, based on the SEM image.

### (Production of Honeycomb Stacked Catalyst)

A honeycomb carrier was wet coated with the Cu-supported AFX-type zeolite obtained of Example A7 so that the percentage of supporting per liter of the honeycomb carrier was 180 g, and thereafter the resultant was subjected to calcination at 500° C. Thus, a honeycomb stacked catalyst of Example A7 was obtained where a catalyst layer including the Cu-supported AFX-type zeolite was provided on the honeycomb carrier.

### Comparative Example A3: Synthesis of CHA-Type Zeolite

To 930.0 g of an aqueous 25% N,N, N-trimethyl adamantane ammonium hydroxide solution (hereinafter, sometimes referred to as “aqueous 25% TMAdaOH solution”) were added 2,080 g of water, 826 g of amorphous synthetic aluminum silicate (synthetic aluminum silicate, trade name: Kyoward (registered trademark) 700PEL manufactured by Kyowa Chemical Industry Co., Ltd., SAR: 10.0), 320.0 g of colloidal silica (trade name: Snowtex (registered trademark) 40 manufactured by Nissan Chemical Corporation, percentage of SiO₂ contained: 39.7%), 133.0 g of 48% sodium hydroxide (manufactured by Kanto Kagaku), and 23.0 g of a Chabazite seed crystal (SAR10), and the resultant was sufficiently mixed, to thereby obtain a starting material composition (mixture). The compositional ratio (molar ratio) in the starting material composition was as follows.

TABLE 10
SiO₂

| | |
|---|---|
| 0.081 | Al₂O₃ |
| 0.100 | TMAdaOH |
| 0.100 | Na₂O |
| 16.00 | H₂O |

The numerical value of each of the components in the mixture means the molar number ratio with the molar number of SiO₂ as 1.

The starting material composition (mixture) was loaded into a 5,000-cc stainless autoclave and sealed, thereafter heated to 160° C. and retained for 48 hours with stirring at 300 rpm, and thereafter retained at 170° C. for 24 hours. A product after this hydrothermal treatment was subjected to solid-liquid separation, and the resulting solid phase was washed with a sufficient amount of water and dried at 105° C., to thereby obtain a product. Powder X-ray diffraction analysis was performed and it was thus confirmed that the product was a single phase of pure CHA-type zeolite. Fluorescent X-ray analysis was performed, and the silica/alumina ratio (SiO₂/Al₂O₃) in the CHA-type aluminosilicate obtained of Comparative Example A3 was here 11.3.

A NH₄⁺-type CHA-type zeolite was obtained from the CHA-type zeolite obtained of Comparative Example A3, in the same manner as in Example A7.

### (Supporting of Cu on CHA Zeolite)

After 120.0 g of the NH₄⁺-type CHA-type zeolite obtained was impregnated with a mixture of 34.0 g of an aqueous 50% copper nitrate trihydrate solution and 30.0 g of water, the resultant was dried at 100 to 120° C. The resultant was impregnated with a mixture of 12.0 g of morpholine and 48.0 g of water under an environment of 25° C., and again dried at 100 to 120° C., to thereby obtain a Cu-supported CHA-type zeolite. The amount of supporting of Cu in terms of solid content, as measured by fluorescent X-ray analysis, was 3.9% by mass, and the SAR (SiO₂/Al₂O₃ ratio) was 11.3. FIG. 18 illustrates an SEM image. The average particle size was about 0.2 μm.

### (Production of Honeycomb Stacked Catalyst)

A honeycomb stacked catalyst of Comparative Example A3 was obtained in the same manner as in Example A7 except that the Cu-supported CHA-type zeolite obtained of Comparative Example A3 was used.

### Comparative Example A4

To 330.0 g of an aqueous 25% TMAdaOH solution were 2,800 g of water, 45.0 g of sodium aluminate (manufactured by FUJIFILM Wako Pure Chemical Corporation), 220.0 g of precipitated silica (trade name: Nipsil (registered trademark) ER manufactured by Tosoh Silica Corporation), 60.0 g of J sodium silicate No. 3 (manufactured by Nippon Chemical Industrial Co., Ltd., content of SiO₂: 29% by mass; content of Na₂O: 9.5% by mass), and 20 g of a Chabazite seed crystal (SAR13), and the resultant was sufficiently mixed, to thereby obtain a starting material composition. The compositional ratio (molar ratio) in the starting material composition was as follows.

TABLE 11
SiO₂

| | |
|---|---|
| 0.065 | Al₂O₃ |
| 0.104 | TMAdaOH |
| 0.100 | Na₂O |
| 44.40 | H₂O |

The numerical value of each of the components in the mixture means the molar number ratio with the molar number of SiO₂ as 1.

The starting material composition was loaded into a 5,000-cc stainless autoclave and sealed, and thereafter heated to 160° C. and retained for 48 hours with stirring at 300 rpm. A product after this hydrothermal treatment was subjected to solid-liquid separation, and the resulting solid phase was washed with a sufficient amount of water and dried at 105° C., to thereby obtain a product. Powder X-ray diffraction analysis was performed and it was thus confirmed that the product was a single phase of CHA zeolite. Fluorescent X-ray analysis was performed and the silica/alumina ratio (SiO₂/Al₂O₃) in the CHA-type aluminosilicate obtained was here 13.4.

A NH₄⁺-type CHA zeolite was obtained by subjecting the CHA-type zeolite obtained of Comparative Example A4, to the same manner as in Example A7.

### (Supporting of Cu on CHA-Type Zeolite)

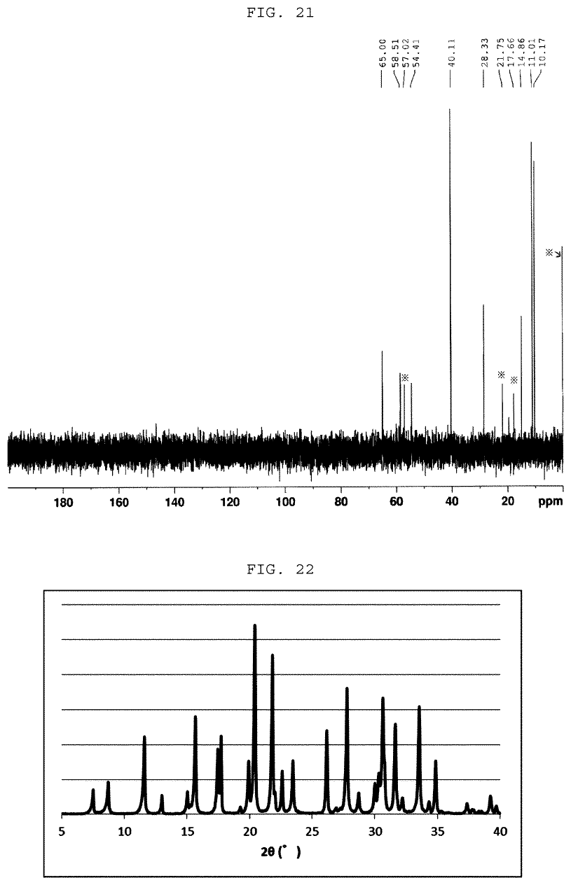

After 160.0 g of the NH₄⁺-type CHA-type zeolite obtained was impregnated with a mixture of 42.0 g of an aqueous 50% copper nitrate trihydrate solution and 42.0 g of water, the resultant was dried at 100 to 120° C., to thereby obtain a Cu-supported CHA-type zeolite. The amount of supporting of Cu in terms of solid content, as measured by fluorescent X-ray analysis, was 4.8% by mass, and the SAR (SiO₂/Al₂O₃ ratio) was 13.4. FIG. 19 illustrates an SEM image. The average particle size was about 0.3 μm. In this regard, the primary particle size was as finer as 0.1 μm or less.

### (Production of Honeycomb Stacked Catalyst)

A honeycomb stacked catalyst of Comparative Example A4 was obtained in the same manner as in Example A7 except that the Cu-supported CHA-type zeolite obtained of Comparative Example A4 was used.

### <Laboratory Measurement of Reduction Efficiency of Nitrogen Oxide>

The hydrothermal durability was measured by cutting each of the honeycomb stacked catalysts of Example A7, Comparative Example A3 and Comparative Example A4 into a cylinder shape of 25.4 mmφ diameter×50 mm length, placing the resultant in an electric furnace (trade name OXK-600X, manufactured by Kyoei Electric Kilns Co., Ltd.) to which a gas humidification apparatus (trade name RMG-1000, manufactured by J-Science Lab Co., Ltd.) was connected, and retaining the resultant at 650° C. for 100 hours under supply of air including 10% steam at a flow rate of 70 L/min. The sample after measurement of the hydrothermal durability was mounted on a catalyst evaluation apparatus (trade name SIGU-2000, manufactured by Horiba Ltd.), and the compositional ratio in a gas was analyzed by an automobile exhaust gas measurement apparatus (trade name MEXA-6000 FT, manufactured by Horiba Ltd.), and thus the reduction efficiency of nitrogen oxide was measured in a steady flow of model gas. The model gas here used included 210 ppm of NO, 40 ppm of NO₂, 250 ppm of NH₃, 4% of H₂O, 10% of O₂, and N₂ as the balance, and the measurement was performed in the temperature range from 170° C. to 500° C. at a space velocity SV of 59,000 h⁻¹.

The results are shown in Table 12.

TABLE 12

| | Percentage of | | | | |
|---|---|---|---|---|---|
| | cleaning-up | | | | |
| | of NOx | | | | |
| | 200° C. | 500° C. | SAR | Cu % | Cu/Al |
| Example A7 | 91% | 90% | 10.7 | 4.2% | 0.26 |
| Comparative Example A3 | 85% | 88% | 11.3 | 3.9% | 0.25 |
| Comparative Example A4 | 73% | 65% | 13.4 | 4.8% | 0.36 |

The compound of the present invention, even in the form of iodide as it is or in the form of hydroxide, can allow a single phase of AFX-type zeolite to be obtained. In other words, the compound of the present invention is high in performance as OSDA because the compound not only can be subjected to preparation of an AFX-type zeolite with saving of the labor for deriving from iodide to other salt, but also can allow a desired AFX-type zeolite to be acquired as a single substance.

### Production Example B1; Preparation of Pt—V/HAP Catalyst

To 90 mL of acetone were Pt(acac)₂ (platinum acetylacetonate, 0.4 mmol) manufactured by N.E. Chemcat Corporation and VO(acac)₂ (vanadyl acetylacetonate, 0.4 mmol) manufactured by Sigma-Aldrich Co. LLC, and the resultant was stirred at room temperature for 30 minutes. Furthermore, 1.0 g of HAP (trade name “tricalcium phosphate”) of FUJIFILM Wako Pure Chemical Corporation was added and the resultant was stirred at room temperature for 4 hours. The solvent was removed from the resulting mixture by a rotary evaporator, to thereby obtain a light green powder. The powder obtained was dried at 110° C. overnight. Furthermore, the powder dried was pulverized in an agate mortar and calcined in air at 300° C. for 3 hours, to thereby obtain a charcoal powder (Pt—V/HAP).

### Example B1: Synthesis of N,N,N′,N′-tetraethylbicyclo[2.2.2]octane-2,3:5,6-dipyrrolidinium diiodide

(synthesis of N,N′-diethylbicyclo[2.2.2]octane-2,3:5,6-dipyrrolidine)

Into a 50-mL stainless autoclave were added 0.3 g of the Pt—V/HAP obtained in Production Example B1, 0.3 mmol of N,N′-diethylbicyclo[2.2.2]oct-7-ene-2,3:5,6-tetracarbodiimide synthesized according to the method of Patent Literature 2, and 0.1 g of molecular sieves 4 Å of FUJIFILM Wako Pure Chemical Corporation, 5 mL of 1,2-dimethoxyethane (DME) as a solvent was added thereto, and a hydrogenation reaction was performed at a reaction temperature of 150° C. and a hydrogen pressure of 5 MPa for 48 hours. After the reaction, the yield of N,N′-diethylbicyclo[2.2.2]octane-2,3:5,6-dipyrrolidine was measured with GC-MS, and the yield was 77%. A product was isolated and subjected to NMR measurement. The results are shown below.

¹H NMR (400 MHZ, CDCl₃) δ=2.72 (t, J=17 Hz, 4H), 2.49 (dd, J=30, 14 Hz, 4H), 2.43 (dd, J=18, 10 Hz, 4H), 2.21 (s, 4H), 1.57 (s, 4H), 1.40 (s, 2H), 1.14 (t, J=15 Hz, 6H);

¹³C NMR (100 MHZ, CDCl₃) δ=57.0 (×4), 50.2 (×2), 40.7 (×4), 30.6 (×2), 14.6 (×2), 13.9 (×2).

(Synthesis of N,N,N′,N′-tetraethylbicyclo[2.2.2]octane-2,3:5,6-dipyrrolidinium diiodide)

In a 100-mL flask was placed a 50 mL of an ethanol solution of 2.2 g of N,N′-diethylbicyclo[2.2.2]octane-2,3:5,6-dipyrrolidine (molecular weight 248.41) synthesized by the above synthesis, and 6.0 g of ethyl iodide (molecular weight 155.97, liquid, Tokyo Chemical Industry Co., Ltd.) was dropped. After reflux under a nitrogen atmosphere for 2 days, the resultant was cooled and filtrated, and washed with acetone and dried, to thereby obtain 2.6 g of a white powder (yield 52%) of an objective substance, N,N,N′,N′-tetraethylbicyclo[2.2.2]octane-2,3:5,6-dipyrrolidinium diiodide. ¹H-NMR and ¹³C-NMR of the white powder obtained are shown below.

¹H NMR (400 MHZ, D₂O) δ: 3.82 (dd, 4H), 3.49 (q4, 4H), 3.38 (q4, 4H), 3.33 (d, 4H), 2.68 (m, 4H), 1.80 (s, 2H), 1.64 (s, 4H), 1.36 (t, 6H), 1.31 (t, 6H) ¹³C NMR (400 Hz, CDCl₃) δ: 65.00 (×4), 58.51 (×2), 54.41 (×2), 40.11 (×4), 28.33 (×2), 14.86 (×2), 11.01 (×2), 10.1 (×2)

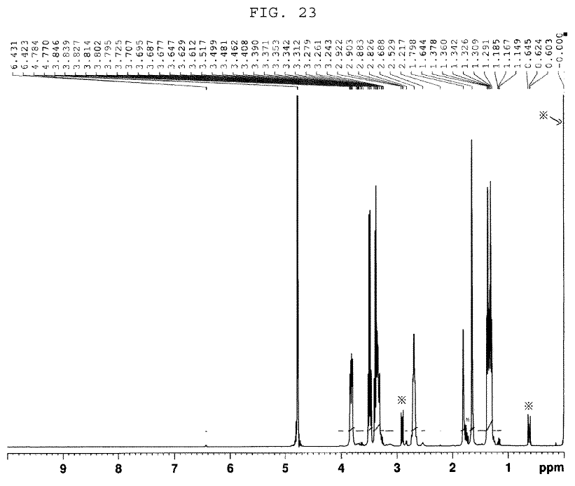

¹HNMR spectral data of N,N,N′,N′-tetraethylbicyclo[2.2.2]octane-2,3:5,6-dipyrrolidinium diiodide is illustrated in FIG. 20 and ¹³CNMR spectral data of N,N,N′,N′-tetraethylbicyclo[2.2.2]octane-2,3:5,6-dipyrrolidinium diiodide is illustrated in FIG. 21.

### Reference Example B1: Synthesis of AFX-Type Zeolite

In a SUS beaker were stirred 2.0 g of N,N,N′,N′-tetraethylbicyclo[2.2.2]octane-2,3:5,6-dipyrrolidinium diiodide (molecular weight 558.62), 8.4 g of a 4.8% by mass sodium hydroxide solution, 2.7 g of FAU-type zeolite CBV712 (manufactured by Zeolyst C.V., silica/alumina ratio SAR: 10.9), and 3.3 g of water for 48 hours. The compositional ratio in the mixture was as follows.

TABLE 13
SiO₂

| | |
|---|---|
| 0.092 | Al₂O₃ |
| 0.106 | OSDA |
| 0.153 | Na₂O |
| 19.98 | H₂O |

The numerical value of each of the components in the mixture means the molar number ratio with the molar number of SiO₂ as 1.

Next, the starting material composition (mixture) was placed in a 50-cc stainless sealed pressure-resistant container with an inner cylinder of Teflon, and left to stand still and retained at 170° C. for 48 hours. A product after this hydrothermal treatment was subjected to solid-liquid separation, and the resulting solid phase was washed with a sufficient amount of water and dried at 105° C., to thereby obtain a product. Powder X-ray diffraction analysis was performed and it was thus confirmed that the product was a single phase of AFX-type zeolite.

FIG. 22 illustrates an XRD data of the AFX-type zeolite.

### Reference Example B2: Synthesis of N,N,N′,N′-tetraethylbicyclo[2.2.2]octane-2,3:5,6-dipyrrolidinium diiodide

In 1,200 mL of an isopropyl alcohol (IPA)-modified alcohol was dissolved 370.0 g of N,N′-diethylbicyclo[2.2.2]oct-7-ene-2,3:5,6-dipyrrolidine (molecular weight 246.39) synthesized according to Patent Literature 2, and 31.08 g (corresponding to 1.0% by mol of a substrate as palladium) of a 5% palladium carbon catalyst (water-containing K-type product manufactured by N.E. Chemcat Corporation), in terms of dry mass, was added thereto to allow a reaction to occur by hydrogen at 50° C. and an ordinary pressure for 190 hours. The percentage of conversion of the substrate according to gas chromatography (GC) was 99% or more. After the catalyst was removed by separation with filtration, 516.0 g (molecular weight 155.11, 2.2 equivalents) of ethyl iodide was dropped with stirring. The resultant was mildly refluxed in a nitrogen atmosphere for 16 hours, thereafter cooled and then filtered, and washed with acetone and dried, to thereby obtain 703.0 g (yield 90%) of a white powder of an objective substance, N,N,N′,N′-tetraethylbicyclo[2.2.2]octane-2,3:5,6-dipyrrolidinium diiodide.

¹H-NMR and ¹³C-NMR of the white powder obtained are shown below.

¹H-NMR (400 MHZ, D₂O) δ: 3.82 (dd, 4H), 3.49 (q4, 4H), 3.38 (q4, 4H), 3.33 (d, 4H), 2.69 (m, 4H), 1.80 (s, 2H), 1.64 (s, 4H), 1.36 (t, 6H), 1.31 (t, 6H).

¹³C-NMR (100 Hz, D₂O) δ: 65.00 (×4), 58.51 (×2), 54.41 (×2), 40.11 (×4), 28.33 (×2), 14.86 (×2), 11.01 (×2), 10.17 (×2)

¹HNMR spectral data of N,N,N′,N′-tetraethylbicyclo[2.2.2]octane-2,3:5,6-dipyrrolidinium diiodide is illustrated in FIGS. 23, and ¹³CNMR spectral data of N,N,N′,N′-tetraethylbicyclo[2.2.2]octane-2,3:5,6-dipyrrolidinium diiodide is illustrated in FIG. 24.

### Reference Example B3: Synthesis of AFX Zeolite

In a polyethylene beaker were stirred 28.0 g of N,N,N′,N′-tetraethylbicyclo[2.2.2]octane-2,3:5,6-dipyrrolidinium diiodide (molecular weight 558.62) of Reference Example B2, 116.0 g of a 4.8% by mass sodium hydroxide solution, 37.5 g of FAU-type zeolite CBV712 (manufactured by Zeolyst C.V., silica/alumina ratio SAR: 10.9), and 47.0 g of water for 48 hours. The compositional ratio in the mixture was as follows.

TABLE 14
SiO₂

| | |
|---|---|
| 0.092 | Al₂O₃ |
| 0.107 | OSDA |
| 0.152 | Na₂O |
| 19.94 | H₂O |

The numerical value of each of the components in the mixture means the molar number ratio with the molar number of SiO₂ as 1.

Next, the starting material composition (mixture) was placed in a 300-cc stainless sealed pressure-resistant container with an inner cylinder of Teflon, and left to stand still and retained at 170° C. for 40 hours. A product after this hydrothermal treatment was subjected to solid-liquid separation, and the resulting solid phase was washed with a sufficient amount of water and dried at 105° C., to thereby obtain a product. Powder X-ray diffraction analysis was performed and it was thus confirmed that the product was a single phase of AFX-type zeolite.

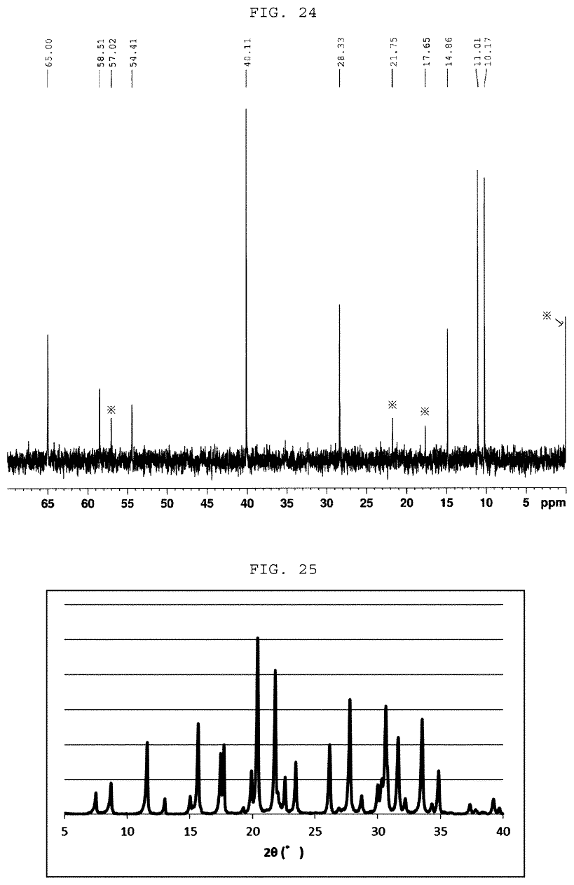

FIG. 25 illustrates XRD data of the AFX-type zeolite.

Conditions of the gas chromatography in Example B and Reference Example B were as follows.
Apparatus name: GCMS-QP2010 (manufactured by Shimadzu Corporation)
Column: SH-Rtx-200 MS manufactured by Shimadzu Corporation
Carrier gas: helium
Total flow rate: 98.9 mL/min
Flow rate in column: 2.56 mL/min
Temperature: the temperature of a column oven was raised from 40° C. to 300° C. at 10° C./min and thereafter retained at 300° C. for 10 minutes.

Measurement conditions of the NMR in Example B and Reference Example B were as follows.

Apparatus name: Ascend 4000 (manufactured by Bruker Japan K.K.)

Measurement method: ¹HNMR and ¹³CNMR were measured by dissolving a sample in deuterated water.

Measurement conditions of powder X-ray diffraction in Example B and Reference Example B were as follows.

Apparatus name: X'Pert Pro (manufactured by Spectris)

Measurement method: a powdery measurement sample was packed in a grooved glass sample plate container and subjected to measurement. The measurement was performed at a tube voltage of 45 kV and a tube current of 40 mA with a CuKα ray as an X-ray source.

According to the production method of the present invention, there is no need for use of any strong reducing agent whose handling is difficult, for example, any reducing agent having the risk of ignition and the like, and therefore N,N,N′,N′-tetraalkylbicyclo[2.2.2]octane-2,3:5,6-dipyrrolidinium can be safely and easily produced. According to the production method, synthesis can be made in relatively safe conditions, thus facility burden is less caused and large-lot production can be made, and therefore the compound is enhanced in productivity and economic performance.

### Production Example C1; Preparation of Pt—V/HAP Catalyst

To 90 mL of acetone were Pt(acac)₂ (platinum acetylacetonate, 0.4 mmol) manufactured by N.E. Chemcat Corporation and VO(acac)₂ (vanadyl acetylacetonate, 0.4 mmol) manufactured by Sigma-Aldrich Co. LLC, and the resultant was stirred at room temperature for 30 minutes. Furthermore, 1.0 g of HAP (trade name “tricalcium phosphate”) of FUJIFILM Wako Pure Chemical Corporation was added and the resultant was stirred at room temperature for 4 hours. The solvent was removed from the resulting mixture by a rotary evaporator, to thereby obtain a light green powder. The powder obtained was dried at 110° C. overnight. Furthermore, the powder dried was pulverized in an agate mortar and calcined in air at 300° C. for 3 hours, to thereby obtain a charcoal powder (Pt—V/HAP).

### Production Example C2: Synthesis of N,N′-diethylbicyclo[2.2.2]octane-2,3:5,6-dipyrrolidine

Into a 50-mL stainless autoclave were added 0.3 g of the Pt—V/HAP obtained in Production Example C1, 0.3 mmol of N,N′-diethylbicyclo[2.2.2]oct-7-ene-2,3:5,6-tetracarbodiimide synthesized according to the method of Patent Literature 2, and 0.1 g of molecular sieves 4 Å of FUJIFILM Wako Pure Chemical Corporation, 5 mL of 1,2-dimethoxyethane (DME) as a solvent was added thereto, and a hydrogenation reaction was performed at a reaction temperature of 150° C. and a hydrogen pressure of 5 MPa for 48 hours. After the reaction, the yield of N,N′-diethylbicyclo[2.2.2]octane-2,3:5,6-dipyrrolidine was measured with GC-MS, and the yield was 77%. A product was isolated and subjected to NMR measurement. The results are shown below.

¹H NMR (400 MHZ, CDCl₃) δ=2.72 (t, J=17 Hz, 4H), 2.49 (dd, J=30, 14 Hz, 4H), 2.43 (dd, J=18, 10 Hz, 4H), 2.21 (s, 4H), 1.57 (s, 4H), 1.40 (s, 2H), 1.14 (t, J=15 Hz, 6H);

¹³C NMR (100 MHZ, CDCl₃) δ=57.0 (×4), 50.2 (×2), 40.7 (×4), 30.6 (×2), 14.6 (×2), 13.9 (×2).

### Production Example C3: Synthesis of N,N,N′,N′-tetraethylbicyclo[2.2.2]octane-2,3:5,6-dipyrrolidinium diiodide

In a 100-mL flask was placed a 50 mL of an ethanol solution of 2.2 g of N,N′-diethylbicyclo[2.2.2]octane-2,3:5,6-dipyrrolidine (molecular weight 248.41) synthesized according to Production Example C2, and 6.0 g of ethyl iodide (molecular weight 155.97, liquid, Tokyo Chemical Industry Co., Ltd.) was dropped. After reflux under a nitrogen atmosphere for 2 days, the resultant was cooled and filtrated, and washed with acetone and dried, to thereby obtain 2.6 g of a white powder (yield 52%) of an objective substance, N,N,N′,N′-tetraethylbicyclo[2.2.2]octane-2,3:5,6-dipyrrolidinium diiodide. ¹HNMR and ¹³CNMR of the white powder obtained are shown below.

¹H NMR (400 MHZ, D₂O) δ: 3.82 (dd, 4H), 3.49 (q4, 4H), 3.38 (q4, 4H), 3.33 (d, 4H), 2.68 (m, 4H), 1.80 (s, 2H), 1.64 (s, 4H), 1.36 (t, 6H), 1.31 (t, 6H)

¹³C NMR (400 Hz, CDCl₃) δ: 65.00 (×4), 58.51 (×2), 54.41 (×2), 40.11 (×4), 28.33 (×2), 14.86 (×2), 11.01 (×2), 10.1 (×2)

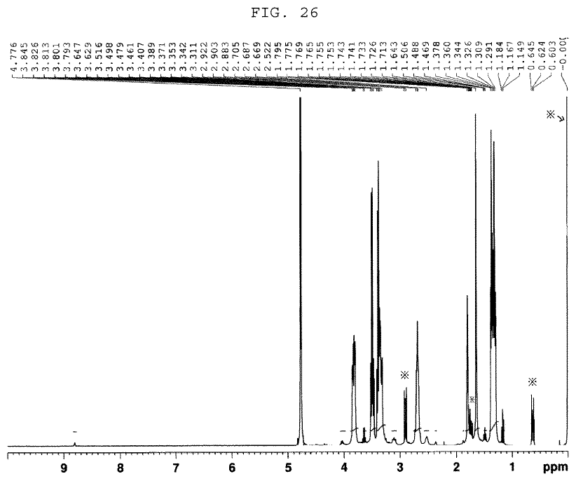

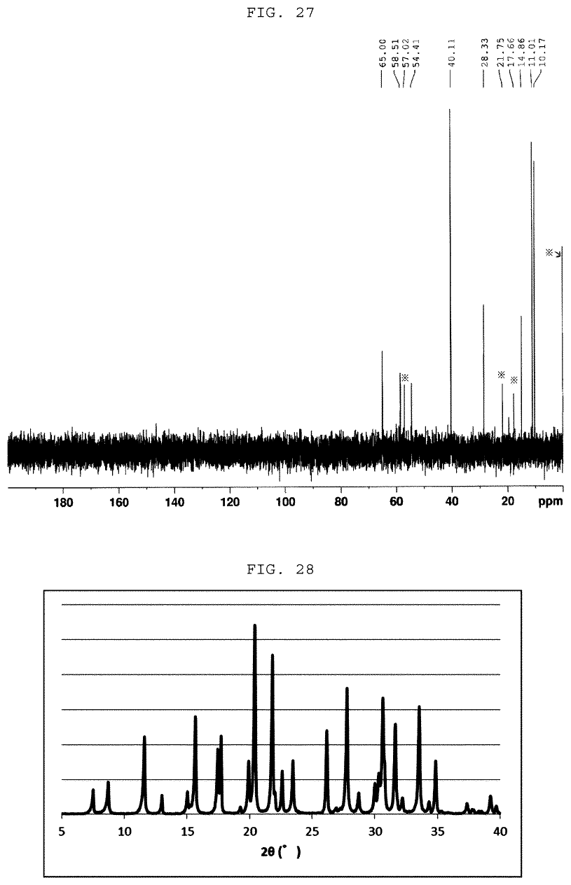

¹HNMR spectral data is illustrated in FIG. 26 and ¹³CNMR spectral data is illustrated in FIG. 27.

### Example C1: Synthesis of AFX-Type Zeolite

In a SUS beaker were stirred 2.0 g of N,N,N′,N′-tetraethylbicyclo[2.2.2]octane-2,3:5,6-dipyrrolidinium diiodide (molecular weight 558.62), 8.4 g of a 4.8% by mass sodium hydroxide solution, 2.7 g of FAU-type zeolite CBV712 (manufactured by Zeolyst C.V., silica/alumina ratio SAR: 10.9), and 3.3 g of water for 48 hours. The compositional ratio in the mixture was as follows.

TABLE 15
SiO₂

| | |
|---|---|
| 0.092 | Al₂O₃ |
| 0.106 | OSDA |
| 0.153 | Na₂O |
| 19.98 | H₂O |

The numerical value of each of the components in the mixture means the molar number ratio with the molar number of SiO₂ as 1.

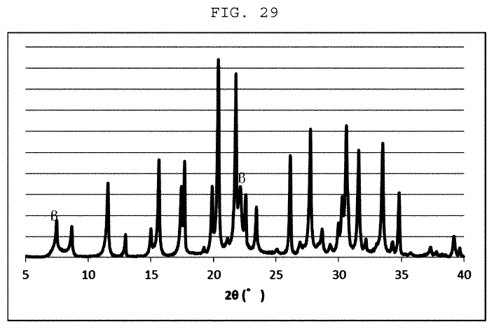

Next, the starting material composition (mixture) was placed in a 50-cc stainless sealed pressure-resistant container with an inner cylinder of Teflon, and left to stand still and retained at 170° C. for 48 hours. A product after this hydrothermal treatment was subjected to solid-liquid separation, and the resulting solid phase was washed with a sufficient amount of water and dried at 105° C., to thereby obtain a product. Powder X-ray diffraction analysis was performed and it was thus confirmed that the product was a single phase of AFX-type zeolite. FIG. 28 illustrates an XRD chart of the AFX-type zeolite obtained by Example C1.

### Comparative Example C1: Synthesis of AFX-Type Zeolite

A product was obtained in the same manner as in Example C1 except that 2.0 g of N,N,N′,N′-tetraethylbicyclo[2.2.2]oct-7-ene-2,3:5,6-dipyrrolidinium diiodide (molecular weight 556.61) synthesized according to the method of Patent Literature 2 was used instead of 2.0 g of N,N,N′,N′-tetraethylbicyclo[2.2.2]octa-2,3:5,6-dipyrrolidinium diiodide (molecular weight 558.62). Powder X-ray diffraction analysis was performed and it was thus confirmed that not only an AFX-type zeolite, but also a beta-type zeolite was produced in the product. FIG. 29 illustrates an XRD chart of the AFX-type zeolite obtained by Comparative Example C1.

According to the production method of the present invention, a single phase of AFX-type zeolite can be obtained even if OSDA is in the form of iodide as it is, and the method is useful. An unsafe reducing agent such as lithium aluminum hydride (LiAlH₄) can also be avoided from being used in mass production of an AFX-type zeolite.

Diffraction peaks obtained as the results of powder X-ray diffraction analysis of the AFX-type zeolite produced in Example C1 are shown in the following Table.

TABLE 16
Example C1
2θ(°)

7.50
8.72
11.60
13.02
15.69
17.46
17.73
19.92
20.42
21.84
23.47
26.18
27.80
30.66
31.64
33.56

### INDUSTRIAL APPLICABILITY

According to the present invention, supply of, for example, a zeolite which is useful as a material of OSDA and which is, for example, one of water-containing aluminosilicates can be realized in a relatively stable manner at low cost. According to the production method of the present invention, a compound serving as a material of OSDA can be simply and safely provided, and supply of, for example, a zeolite which is one of water-containing aluminosilicates can be realized in a relatively stable manner at low cost. According to the present invention, supply of, for example, an AFX-type zeolite which is one of water-containing aluminosilicates can be realized in a relatively stable manner at low cost, and nitrogen oxide can be cleaned up by use of a reducing component at a high efficiency according to an aspect of, for example, a honeycomb stacked catalyst where a honeycomb carrier is coated with, for example, an AFX-type zeolite.

Therefore, the present invention can be widely and effectively utilized in applications of not only various inorganic or organic molecular adsorbents or separation agents, but also, for example, drying agents, dehydration agents, ion-exchangers, petroleum refining catalysts, petrochemical catalysts, solid acid catalysts, ternary catalysts, catalysts for cleaning up exhaust gases, and NOx occlusion materials.

1. A method to prepare a compound of formula (1), or a salt thereof, comprising:
providing a compound represented by formula (A);
reacting the compound of formula (A) with a hydrogen source and a catalyst comprising platinum, to obtain a compound of formula (2); and
N-alkylating the compound of formula (2) with an alkylation reagent, 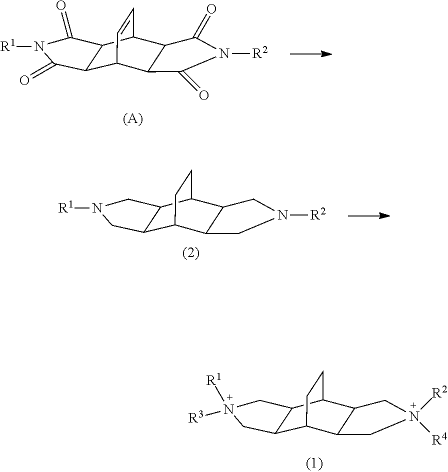
wherein R¹ to R⁴ are each independently an alkyl group.

2. The method according to claim 1, wherein
the catalyst further comprises vanadium.

3. The method according to claim 1, wherein
the catalyst further comprises a carrier selected from the group of inorganic oxides consisting of silica, titania, zirconia, alumina, magnesium oxide, tricalcium phosphate, and any composite of two or more thereof.

4. The method according to claim 2, wherein
the catalyst further comprises a carrier selected from the group of inorganic oxides consisting of silica, titania, zirconia, alumina, magnesium oxide, tricalcium phosphate, and any composite of two or more thereof.

5. The method according to claim 1, wherein
the catalyst further comprises tricalcium phosphate.

6. The method according to claim 2, wherein
the catalyst further comprises tricalcium phosphate.
